SEQUENCE LISTING (1 sequence)

SEQ ID NO: 1 - protein, 29 aa, Homo sapiens
HSQGTFTSDYSKYLDSRRAQDFVQWLMNT

(19) United States
(12) Patent Application Publication
(10) Pub. No.: US 2025/0276127 A1
(43) Pub. Date: Sep. 4, 2025

(54) THERAPEUTIC METHODS AND DEVICES

(71) Applicant: NORWEGIAN UNIVERSITY OF SCIENCE AND TECHNOLOGY (NTNU), Trondheim (NO)

(72) Inventors: Sven Magnus CARLSEN, Tiller (NO); Sverre Christian CHRISTIANSEN, Sjetnemarka (NO); Anders Lyngvi FOUGNER, Trondheim (NO); Øyvind STAVDAHL, Trondheim (NO); Reinold ELLINGSEN, Ranheim (NO); Dag Roar HJELME, Trondheim (NO)

(73) Assignee: NORWEGIAN UNIVERSITY OF SCIENCE AND TECHNOLOGY (NTNU), Trondheim (NO)

(21) Appl. No.: 18/572,594

(22) PCT Filed: Jun. 23, 2022
(86) PCT No.: PCT/EP2022/067148

§ 371 Date: Dec. 20, 2023

(30) Foreign Application Priority Data
Jun. 24, 2021 (GB) 2109087.3

(51) Int. Cl. A61M 5/172; A61B 5/145; A61B 5/1486; A61K 38/26; A61K 38/28

(52) U.S. Cl. CPC A61M 5/1723; A61B 5/14532; A61B 5/14865; A61K 38/26; A61K 38/28; A61M 2005/1726

(57) ABSTRACT

The present invention relates to a novel medical use of glucagon, and other compounds with glucagon activity, as vasodilators, to assist in the delivery of therapeutic agents or in the operation of sensor devices which determine the level of analytes in the blood. In particular, the compound is administered in temporal coordination with the active agent and/or in conjunction with determination of the analyte by a bodily sensor, and at a site which is in the vicinity of the site of administration of the active agent and/or of the site(s) of sensing of the analyte by the bodily sensor. This includes in particular the delivery of insulin in the treatment of diabetes, and the determination of blood glucose levels by glucose sensors. Also provided herein are integrated systems for performing the medical uses and therapies.

### FIELD

The present disclosure, and the present invention, relates generally to medical uses and therapies, and more particularly to a novel medical use of glucagon, and other compounds with glucagon activity. Compounds with glucagon activity are proposed herein for use as vasodilators, to assist in the delivery of therapeutic agents or in the operation of sensor devices which determine the level of analytes in the blood. This includes in particular the delivery of insulin in the treatment of diabetes, and the determination of blood glucose levels by glucose sensors. Also provided herein are devices for performing the medical uses and therapies.

### BACKGROUND

The treatment and management of subjects with diabetes, most notably type 1 diabetes (T1D), has been an on-going challenge for a long time. The benefits of tight control of blood glucose levels for reducing the microvascular complications of diabetes, and indeed mortality in diabetes, are recognised, but this needs to be balanced with the increased risk of hypoglycaemia and the elevated burden of self- management that accompanies intensive glucose control. To this end, advances in continuous glucose monitoring (CGM) technology and insulin infusion pumps are leading towards the development of an artificial pancreas (AP), an automated system which combines CGM with an insulin pump and an insulin-dosing algorithm to automatically and continuously regulate insulin delivery to control the blood glucose level. Both single hormone (insulin only) and dual-hormone systems (insulin and glucagon) are being developed (Peters and Haidar, 2018, Diabet. Med. 35, 450-459). In dual hormone AP systems glucagon is used for its hormonal effect to increase glucose in the blood, for control of hypoglycaemia.

Whilst such developments hold much promise to improve the management of diabetes, there is an ongoing need for improvements. In particular, it has proved difficult fully to automate sub-cutaneous (SC) AP systems, due to slow dynamics, including particularly the marked delay in the glucose-lowering response of sub-cutaneously administered insulin; it can take 1-2 hours for a major effect on blood glucose levels to be seen. In addition, there is typically a delay of at least 6-8 minutes in sub-cutanenous glucose sensing, although this can be much longer in some subjects. These delays have impeded the development of SC AP systems, and so far only hybrid systems are available where the users inform the system about the amount of carbohydrates ingested, and the system translates that to a dose (a meal bolus) of insulin. For a truly autonomous AP, a rapid effect of insulin on glucose levels is needed after meals and during and after physical activity, and this means the delays in glucose sensing and effect of SC-delivered insulin need to be reduced.

To try to increase dynamics and achieve a faster insulin effect in response to rising blood glucose levels, efforts are being made to develop intra-peritoneal AP systems, since intraperitoneal (IP) delivery of insulin typically results in a quicker response, and indeed better glucose control. This includes the context of a dual-hormone AP (Å m et al., Scientific Reports, 2020, 10, 13735). However, developing an IP AP is technically challenging and new approaches to insulin delivery and glucose sensing are continually being sought.

The present developments are directed towards this need and are based on the vasodilatory effects of glucagon. Glucagon has previously been reported to have vasodilatory activity, but this has been at the level of large blood vessels, namely the aorta (Sélley et al., Horm. Metab. Res. 2016, 48, 476-483), and a therapeutic utilisation or harnessing of this effect has not previously been proposed.

### SUMMARY

To address the issue of delays in insulin effect and glucose sensing, particularly in the context of SC insulin delivery and glucose sensing, it is proposed herein to use the hormone glucagon, or more generally a compound with glucagon activity, as a local vasodilator, to increase blood flow at the location of insulin administration and/or of glucose sensing. This acts to improve the dynamics of the administered insulin and the glucose sensing, and may reduce the delay in insulin effect and improve the performance of the glucose sensor, to achieve a more precise and/or faster determination of blood glucose levels, when needed, for example when glucose levels are changing at times of a meal, or exercise.

According to this new proposal, the glucagon is used not for its usual hormonal effect to counteract, or to prevent episodes of low blood glucose levels, but rather for its vasodilatory effect. The present inventors have surprisingly found that glucagon when administered sub-cutaneously is able to exert a vasodilatory effect at a local level on small blood vessels in the skin, increasing blood flow at the site of administration by several hundred percent. Thus, we have observed a vasodilatory effect at the capillary level, that is in the subcutaneous microcirculation. It is believed that this local effect on small blood vessels at the site of administration will also be seen at other sites, including particularly IP sites of administration. Thus, glucagon, and compounds with glucagon activity, may be used more generally to improve local blood flow at sites of administration and thereby may be used to assist, or enhance, the delivery of therapeutic agents in general, and not just insulin. Similarly, glucagon-active compounds may be used to improve local blood flow at the sites of operation of a sensor for any blood analyte, not just glucose.

Accordingly, in a first and broad aspect provided herein is a compound with glucagon activity for use in the delivery of an active therapeutic agent to a subject and/or in the determination of the blood level of an analyte in the subject, wherein the compound is administered with the active agent and/or in conjunction with determination of the analyte by a bodily sensor, and wherein the compound is administered to the subject at a site which is in the vicinity of the site of administration of the active agent and/or of the site(s) of sensing of the analyte by the sensor, and in temporal coordination with the administration of the active agent and/or with sensing of the analyte by the sensor.

As noted above, a compound with glucagon activity is an active agent in its own right, and hence the active therapeutic agent with which it is co-administered may thus be regarded as a second active agent.

In one embodiment provided herein is compound with glucagon activity for use in the delivery of a second active therapeutic agent to a subject, wherein said use comprises co-administering the compound with said second agent, and said compound is administered to the subject in temporal coordination with the second agent at a site which is in the vicinity of the administration site of the second agent.

It will be understood that the delivery of an active therapeutic agent is made in the context of the therapeutic use of that agent. Accordingly, the compound with glucagon activity is used in the delivery of a (second) therapeutic agent in the treatment and/or prevention of a medical condition which is responsive to that agent. In other words, the compound with glucagon activity is used in the treatment and/or prevention of a condition by the (second) therapeutic agent.

In another embodiment, provided herein is a compound with glucagon activity for use in conjunction with a bodily sensor in the determination of the blood level of an analyte in a subject, said use comprising administering said compound at a site in the vicinity of the sensor in temporal coordination with the time of analyte sensing by the sensor.

As will be explained further below, in an embodiment the administration may take place each time sensing by the sensor occurs (i.e. each time the sensor performs the sensing). In another embodiment, the administration may take place continuously or periodically during the time that sensing occurs, or over a time period during which sensing by the sensor takes place. In this way, the compound may be administered such that it is present in the vicinity of the sensor during the time that sensing by the sensor is performed. For example, the compound may be administered from a controlled release (“slow release”) depot or reservoir (or any other slow release formulation or preparation) positioned or applied in the vicinity of the sensor, or provided as part of the sensor.

It will be understood from the above that “in temporal coordination”, whether in relation to administration of an active agent or in relation to sensing by a sensor means that the administration of the compound is coordinated with the administration of the therapeutic active agent or with the sensing by the sensor, or more generally is such that the compound is present or effective (i.e. active, or able to exert an effect) in the vicinity of the administration site of the active agent or in the vicinity of the sensing site at the time that the administration and/or absorption of the active agent or the sensing occurs. This is discussed further below.

For such uses the compound with glucagon activity may be provided in the form of a composition comprising the compound. In particular embodiments the composition may be referred to as a pharmaceutical composition. In certain embodiments the composition may comprise one or more pharmaceutically-acceptable carriers or excipients.

In a second broad aspect, provided herein is a method of delivering a therapeutic active agent to a subject and/or of determining the level of an analyte in the blood of a subject with a bodily sensor, said method comprising administering to the subject a compound with glucagon activity together with the therapeutic active agent and/or in conjunction with determination of the analyte by a bodily sensor, wherein the compound is administered at a site in the vicinity of the administration site of the active agent or in the vicinity of the sensor, and in temporal coordination with the administration of the second active agent and/or with the time of sensing of the analyte by the sensor.

In one embodiment the method is a method of delivering a therapeutic active agent to a subject, said method comprising co-administering to said subject a compound with glucagon activity together with the therapeutic active agent, wherein the compound is administered at a site in the vicinity of the insulin administration site and in temporal coordination with the second active agent.

As noted above, the method may be a method of treating and/or preventing a condition which is responsive to the therapeutic active agent.

In another embodiment, the method is a method for determining the level of an analyte in the blood of a subject with a bodily sensor, said method comprising administering to the subject a compound with glucagon activity, wherein the compound is administered at a site in the vicinity of the sensor in temporal coordination with the time of analyte sensing by the sensor.

In a third aspect provided herein is the use of a compound with glucagon activity in the manufacture of a pharmaceutical product for use in the delivery of an active therapeutic agent to a subject and/or in the determination of the blood level of an analyte in the subject, wherein the pharmaceutical product comprises the active agent and/or is administered in conjunction with determination of the analyte by a bodily sensor, and wherein the compound is administered to the subject at a site which is in the vicinity of the site of administration of the active agent and/or of the site(s) of sensing of the analyte by the sensor and in temporal coordination with the administration and/or with sensing of the analyte by the sensor.

Where the pharmaceutical product is for use in the delivery of an active therapeutic agent, the compound may be provided, or formulated, together with the active agent in a single composition or preparation, or the compound and active agent may be provided, or formulated, separately, in separate compositions or preparations. Thus, the pharmaceutical product may take the form of a composition comprising both the compound and the active agent, or it may take the form a kit comprising (i) the compound and (ii) the active agent.

The pharmaceutical product may be provided for use in any aspect of the invention herein. The pharmaceutical product comprises the compound and the active agent for simultaneous, separate or sequential administration to the subject, but within the constraint that the administration of the compound is in temporal coordination with the administration of the active agent, as described and defined further below.

In the various aspects and embodiments above, and those further described below and elsewhere herein, the compound with glucagon activity acts to improve local blood flow at the site of its administration. By virtue of the improved blood flow the compound may thereby improve the effect, or the delivery, of the active agent and/or improve the determination of the blood level of the analyte in the subject by the sensor. In an embodiment, the absorption of the active agent may be improved. In an embodiment, the performance of the sensor may be improved. For example, the time taken for determination of the blood analyte level may be reduced.

In a related aspect provided herein is a sensor system for determining the level of an analyte in the blood of a subject, said sensor system comprising:
(i) a sensor configured to determine the blood level of the analyte in the subject and to provide sensor data associated with the blood analyte level;
(ii) a compound delivery means configured to administer a compound with glucagon activity to said subject;
(iii) a control system configured to receive sensor data from the sensor; wherein:
(a) the delivery means comprises a slow-release reservoir of the compound with glucagon activity, which is configured to administer the compound to a site in the vicinity of the sensor; or
(b) the delivery means is controllable to administer the compound with glucagon activity to said subject, and the control system is configured to control the delivery device to administer the compound to a site in the vicinity of the sensor in temporal coordination with operation of the sensor to determine the blood analyte level.

In another related aspect, provided herein is a delivery system for the administration of an active therapeutic agent to a subject, said delivery system comprising:
(i) a compound delivery means configured to administer a compound with glucagon activity to said subject;
(ii) a delivery device configured to administer the active therapeutic agent to said subject; wherein:
(a) the compound delivery means comprises a slow-release reservoir of the compound with glucagon activity, which is configured to administer the compound to a site in the vicinity of the administration site of the active therapeutic agent; or
(b) the compound delivery means is controllable to administer the compound with glucagon activity to said subject, and the delivery system further comprises a control system configured to control the compound delivery means to administer the compound to a site in the vicinity of the active agent administration site in temporal coordination with the administration of the active agent.

In particular embodiments the therapeutic active agent is insulin and the compound and insulin are co-administered in the treatment of diabetes, particularly type 1 diabetes. Thus, the compound is used in the delivery of insulin. Particularly, the compound is used in such embodiments to enhance, or improve, the effect, or the delivery, of insulin.

Further, in particular embodiments the analyte is glucose and the sensor is a glucose sensor. More particularly, in such embodiments the compound is used to improve the determination of the blood glucose level of the subject by the glucose sensor.

Accordingly, in a more particular aspect, there is provided a compound with glucagon activity for use in the treatment and/or management of a subject with diabetes by co-administration with insulin and/or in conjunction with glucose sensing, wherein the compound is administered to the subject at a site which is in the vicinity of the site of insulin administration and/or of the site(s) of glucose sensing by a bodily glucose sensor and in temporal coordination with the insulin administration and/or with the glucose sensing.

In an embodiment, this aspect provides a compound with glucagon activity for use in the delivery of insulin to a subject, wherein said use comprises co-administering the compound with insulin, and said compound is administered to the subject in temporal coordination with the insulin at a site which is in the vicinity of the insulin administration site.

In another embodiment, this aspect provides a compound with glucagon activity for use in conjunction with a bodily glucose sensor in the determination of the blood glucose level of a subject, said use comprising administering said compound at a site in the vicinity of the glucose sensor in temporal coordination with the time of glucose sensing by the sensor.

In a related aspect, also provided herein is a method of treating and/or managing a subject with diabetes, said method comprising co-administering a compound with glucagon activity to the subject at a site which is in the vicinity of a site of insulin administration and/or of a site of glucose sensing by a bodily glucose sensor and in temporal coordination with the insulin administration and/or with the glucose sensing.

In an embodiment this aspect provides a method of delivering insulin to a subject, said method comprising co-administering to said subject a compound with glucagon activity together with insulin, wherein the compound is administered at a site in the vicinity of the insulin administration site and in temporal coordination with the second active agent.

In another embodiment, this aspect provides a method for determining the level of glucose in the blood of a subject with a bodily glucose sensor, said method comprising administering to the subject a compound with glucagon activity, wherein the compound is administered at a site in the vicinity of the glucose sensor in temporal coordination with the time of glucose sensing by the sensor.

A still further aspect provides use of a compound with glucagon activity in the manufacture of a pharmaceutical product for use in treatment and/or management of a subject with diabetes by co-administration with insulin and/or in conjunction with glucose sensing, wherein the pharmaceutical product comprises insulin and/or is administered in conjunction with determination of glucose by a bodily sensor, and wherein the compound for administration to the subject at a site which is in the vicinity of the site of administration of the active agent and/or of the site(s) of glucose sensing by the sensor and in temporal coordination with the insulin administration and/or with the glucose sensing.

As noted above, in this aspect where the pharmaceutical product is for delivery of insulin, the pharmaceutical product may comprise the insulin and the compound in the same or in separate compositions or formulations, and includes a kit, as discussed above.

Also provided herein is an integrated system for controlling the blood glucose level in a subject with diabetes comprising:
(i) one or more glucose sensors configured to determine the blood glucose level of the subject and to provide sensor data associated with the blood glucose level;
(ii) a compound delivery means configured to administer a compound with glucagon activity to said subject;
(iii) an insulin delivery device configured to administer insulin to said subject;
(iv) a control system configured to receive sensor data from the glucose sensor(s) and to
determine a dose of insulin to administer to the subject based at least on the sensor data and to control the insulin delivery device to administer said dose;
wherein:
(a) the compound delivery means comprises a slow-release reservoir of the compound with glucagon activity, which is configured to administer the compound to a site in the vicinity of the insulin administration site and/or in the vicinity of the glucose sensor; or
(b) the compound delivery means is controllable to administer the compound with glucagon activity to said subject, and the control system is configured to control the compound delivery means to administer the compound to a site in the vicinity of the insulin administration site in temporal coordination with the administration of the insulin, thereby to improve blood flow in the vicinity of the insulin administration site, and/or to control the compound delivery means to administer the compound to a site in the vicinity of a glucose sensor in temporal coordination with operation of the glucose sensor to determine the blood glucose level, thereby to improve blood flow in the vicinity of the glucose sensor.

Administration of the compound to a site “in the vicinity” of the insulin administration site and/or “in the vicinity” of the glucose sensor may comprise administration of the compound within 3 cm or 2.5 cm of the insulin administration site and/or administration of the compound within 3 cm or 2.5 cm of the glucose sensor (particularly the site of sampling/sensing on the glucose sensor) and may comprise administration of the compound in close proximity to the insulin administration site and/or glucose sensor (and in particular in close proximity to the site of sampling/sensing on the glucose sensor), wherein close proximity means within 2 cm, for example within 1.5 cm, or within 1 cm.

The compound delivery means of (a) may be any of the slow-release means discussed above, including for example a transdermal patch, a slow-release coating, or a depot formulation. The compound delivery means of (a) may alternatively comprise a compound infusion line connected to a pump in communication with a reservoir comprising the compound, wherein the compound delivery means is configured to provide continuous or near-continuous administration of the compound.

The compound delivery means of (b) may comprise a compound infusion line connected to a pump in communication with a reservoir comprising the compound. The compound delivery means may be controlled to administer the compound in consideration of the timings of the administration(s) and/or sensing. Alternatively, the compound delivery means of (b) may be configured to provide repeated or multiple administrations of the compound to create and maintain a high local concentration of the compound in the vicinity, such that the compound is present at the time that the administration and/or absorption of the insulin or the sensing takes place. In an embodiment, the control system is further configured to control the compound delivery means to administer the compound at a site in the vicinity of the insulin administration site in temporal coordination with the administration of insulin.

Additionally provided herein is a sensor system for determining the level of glucose in the blood of a subject, said sensor system comprising:

(i) a glucose sensor configured to determine the blood glucose level of glucose of the subject and to provide sensor data associated with the blood glucose level;

(ii) a delivery means configured to administer a compound with glucagon activity to said subject;

(iii) a control system configured to receive the sensor data from the glucose sensor; wherein:
(a) the delivery means comprises a slow-release reservoir of the compound with glucagon activity, which is configured to administer the compound to a site in the vicinity of the glucose sensor, thereby to improve blood flow to the vicinity of the glucose sensor; or
   (b) the delivery means is controllable to administer the compound with glucagon activity to said subject, and the control system is configured to control the delivery means to administer the compound to a site in the vicinity of the glucose sensor in temporal coordination with operation of the glucose sensor to measure the blood glucose level, thereby to improve blood flow to the vicinity of the glucose sensor.

Administration of the compound to a site “in the vicinity” of the glucose sensor may comprise administration of the compound within 3 cm or 2.5 cm of the glucose sensor (particularly the site of sampling/sensing on the glucose sensor) and may comprise administration of the compound in close proximity to the glucose sensor (and in particular in close proximity to the site of sampling/sensing on the glucose sensor), wherein close proximity means within 2 cm, for example within 1.5 cm, or within 1 cm.

The compound delivery means of (a) may be any of the slow-release means discussed above, including for example a transdermal patch, a slow-release coating, or a depot formulation. The compound delivery means of (a) may alternatively comprise a compound infusion line connected to a pump in communication with a reservoir comprising the compound, wherein the compound delivery means is configured to provide continuous or near-continuous administration of the compound.

The compound delivery means of (b) may comprise a compound infusion line connected to a pump in communication with a reservoir comprising the compound. The compound delivery means may be controlled to administer the compound in consideration of the timings of the sensing. Alternatively, the compound delivery means of (b) may be configured to provide repeated or multiple administrations of the compound to create and maintain a high local concentration of the compound in the vicinity, such that the compound is present at the time that the sensing takes place.

Further provided herein is an insulin delivery system for the administration of insulin to a subject, said delivery system comprising:
(i) a compound delivery means configured to administer a compound with glucagon activity to said subject;
(ii) an insulin delivery device configured to administer insulin to said subject;
(iii) a control system configured to
   determine a dose of insulin to administer to the subject and to control the insulin delivery device to administer said dose;
wherein:
(a) the compound delivery means comprises a slow-release reservoir of the compound with glucagon activity, which is configured to administer the compound to a site in the vicinity of the insulin administration site; or
(b) the compound delivery means is controllable to administer the compound with glucagon activity to said subject, and the control system is configured to control the compound delivery means to administer the compound to a site in the vicinity of the insulin administration site in temporal coordination with the administration of the insulin.

Administration of the compound to a site “in the vicinity” of the insulin administration site may comprise administration of the compound within 3 cm or 2.5 cm of the insulin administration site, and may comprise administration of the compound in close proximity to the insulin administration site, wherein close proximity means within 2 cm, for example within 1.5 cm, or within 1 cm.

The compound delivery means of (a) may be any of the slow-release means discussed above, including for example a transdermal patch, a slow-release coating, or a depot formulation. The compound delivery means of (a) may alternatively comprise a compound infusion line connected to a pump in communication with a reservoir comprising the compound, wherein the compound delivery means is configured to provide continuous or near-continuous administration of the compound.

The compound delivery means of (b) may comprise a compound infusion line connected to a pump in communication with a reservoir comprising the compound. The compound delivery means may be controlled to administer the compound in consideration of the timings of the administration(s). Alternatively, the compound delivery means of (b) may be configured to provide repeated or multiple administrations of the compound to create and maintain a high local concentration of the compound in the vicinity, such that the compound is present at the time that the administration and/or absorption of the insulin takes place.

### BRIEF DESCRIPTION OF THE DRAWINGS

The devices depicted in FIGS. 4-13 can also be used as an integrated part of an artificial pancreas.

FIG. 1 presents an outline schematic of a SC CGM device.

FIG. 2 presents a flow chart showing the operation of an insulin-only artificial pancreas.

FIG. 3 presents a flow chart showing the operation of a bi-hormonal artificial pancreas.

FIG. 4 presents an outline schematic of a SC CGM with slow-release glucagon in the vicinity of the sensing site.

FIG. 5 presents an outline schematic of a SC CGM with glucagon at the skin surface just above the sensing site.

FIG. 6 presents an outline schematic of a SC CGM with a delivery line for glucagon in the vicinity of the sensing site.

FIG. 7 presents an outline schematic of a SC CGM with one delivery line for glucagon and one delivery line for insulin that are united shortly before ending in the vicinity of the sensing site.

FIG. 8 presents an outline schematic of a SC CGM with alternative ways for delivery of glucagon and insulin to the vicinity of the sensing site (A) separate glucagon and insulin lines; and (B) separate glucagon and insulin delivery lines which become united.

FIG. 9 presents an outline schematic of a delivery line for insulin with glucagon at the skin surface just above the insulin delivery site.

FIG. 10 presents an outline schematic of an insulin delivery device with slow release glucagon at the tip of the delivery line.

FIG. 11 presents an outline schematic of a SC CGM with an insulin delivery line and with glucagon at the skin surface just above the sensing and insulin delivery site.

FIG. 12 presents an outline schematic of a SC CGM with slow release glucagon at the tip of the sensor and an insulin delivery line ending in the vicinity of the sensing and glucagon releasing site.

FIG. 13 presents an outline schematic of a SC CGM and an insulin delivery line coated with slow release glucagon ending in the vicinity of the sensing site.

FIG. 14 is a graph showing blood flow in human subjects after injection of 0.1 mg glucagon or placebo (0.9% saline) at subcutaneous sites on the lateral side of both upper arms, measured by the laser Doppler technique with 95% confidence intervals shown in long dashed line for glucagon and fine dotted line for placebo; blood perfusion units shown over time (minutes).

FIG. 15 is a graph showing blood flow in human subjects after injection of varying doses of glucagon (0.1 mg, 0.015 mg and 0.01 mg) or placebo (0.9% saline) at subcutaneous sites on the lateral side of both upper arms, measured by the laser Doppler technique; blood perfusion units shown over time (minutes).

FIG. 16 is a graph showing blood flow in human subjects after injection of varying doses of glucagon (0.1 mg, 0.015 mg and 0.01 mg) at subcutaneous sites on the lateral side of both upper arms, measured by the laser Doppler technique, with the effect on blood flow caused by placebo (0.9% saline) subtracted; blood perfusion units shown over time (minutes).

FIG. 17 is a graph showing blood flow in human subjects after injection of 0.1 mg glucagon or placebo (0.9% saline) at subcutaneous sites on symmetric sides the abdomen, measured by the laser Doppler technique with 95% confidence intervals shown in long dashed line for glucagon and fine dotted line for placebo; blood perfusion units shown over time (minutes).

FIG. 18 is a graph showing blood flow in human subjects after injection of varying doses of glucagon (0.1 mg, 0.015 mg and 0.01 mg) or placebo (0.9% saline) at subcutaneous sites on both sides of the abdomen, measured by the laser Doppler technique; blood perfusion units shown over time (minutes).

FIG. 19 is a graph showing blood flow in human subjects after injection of varying doses of glucagon (0.1 mg, 0.015 mg and 0.01 mg) at subcutaneous sites on both sides of the abdomen, measured by the laser Doppler technique, with the effect on blood flow caused by placebo (0.9% saline) subtracted; blood perfusion units shown over time (minutes).

FIG. 20 is a graph showing blood flow in human subjects after injection of 0.1 mg glucagon at subcutaneous sites on both sides of the abdomen using either an injection lasting 1 to 3 seconds or an injection lasting at least 10 seconds, measured by the laser Doppler technique; blood perfusion units shown over time (minutes).

FIG. 21 is a graph showing blood flow in human subjects after injection of 0.1 mg glucagon or placebo (0.9% saline) at subcutaneous sites on symmetric sides of the abdomen using either an injection lasting 1 to 3 seconds or an injection lasting at least 10 seconds, measured by the laser Doppler technique; blood perfusion units shown over time (minutes).

FIG. 22 is a graph showing blood flow in human subjects after injection of 0.05 mg glucagon or placebo (0.9% saline) at subcutaneous sites on the thighs, measured by the laser Doppler technique, with 95% confidence intervals shown in long dashed line for glucagon and fine dotted line for placebo; blood perfusion units shown over time (minutes).

FIG. 23 is a graph showing blood flow in human subjects after injection of varying doses of glucagon (0.05 mg, 0.03 mg and 0.01 mg) or placebo (0.9% saline) at subcutaneous sites on the thighs, measured by the laser Doppler technique; blood perfusion units shown over time (minutes).

FIG. 24 is a graph showing blood flow in human subjects after injection of varying doses of glucagon (0.05 mg, 0.03 mg and 0.01 mg) at subcutaneous sites on the thighs, measured by the laser Doppler technique, with the effect on blood flow caused by placebo (0.9% saline) subtracted; blood perfusion units shown over time (minutes).

FIG. 25 is a graph showing blood flow in human subjects after injection of 0.015 mg glucagon or placebo (0.9% saline) at subcutaneous sites on the lateral side of both upper arms, measured by the laser Doppler technique, wherein the injections were performed at varying distances from the laser Doppler probe (under probe, 1.6 cm from probe centre, 3 cm from probe centre, 5 cm from probe centre). The effect on blood flow caused by placebo (0.9% saline) subtracted; blood perfusion units shown over time (minutes).

FIG. 26 is a graph showing continuous CGM data in human subjects from 12 meals in a non-diabetic female and 11 meals in a non-diabetic male collected with Dexcom G6. The subjects wore two CGMs placed symmetrically on the lateral side of each overarm. 1-3 minutes before the start of ingesting the meal 0.1 ml of glucagon (1 mg/ml) was injected at the site of one CGM and 0.1 ml of placebo (0.9% saline) on the contralateral site of CGM. Before each meal, the site of glucagon delivery was decided by a new randomization. Glucose levels at the start of each meal was set as zero (baseline and the change from baseline is given in the figure). Values are given as means.

### DETAILED DESCRIPTION

The various aspects presented herein are based on a new application of the vasodilatory property of glucagon, and other compounds having the activity of glucagon. The vasodilatory activity of such compounds increases blood flow at the site of their administration, and this increased local blood flow has benefits for methods and uses which rely on local blood flow for their effects, such as drug delivery and the operation of sensors which determine analytes in blood. Thus, the present inventors have discovered that this vasodilatory activity may be harnessed in the novel use of compounds having glucagon activity in the delivery of active agents to a subject, and/or the determination of the level of an analyte in the blood of a subject.

By administering the compound to the site of administration of an active agent (i.e. a drug), or to the site at which sensing by an analyte sensor takes place, local blood flow at that site is increased. In turn this leads to an improvement in the effect of the administered active agent (for example a faster effect may be seen, or any lag or delay in effect following administration may be reduced, as discussed further below). In this way, the delivery of the active agent may be improved. Similarly, increased local blood flow at the site of sensing by the sensor may improve the performance of the sensor, for example by reducing a delay in the determination of the analyte level in the blood.

In particular, as shown in the Examples below, it has been observed that when glucagon is administered, there is an immediate and large increase in local blood flow. This decreases over time, for example over a period of 30 minutes or so. Significantly, it has been observed that the blood flow returns to a level which is higher than the baseline level before glucagon administration, and this increased level is maintained over a prolonged period. Administration of the same volume of a placebo (physiological saline) had little such acute effect and no long-term effect. Thus, administration of a compound with glucagon activity increases local blood flow in the area of administration over a period of time which is useful to take advantage of the increased blood flow to deliver another active agent, or to operate a sensor.

These findings have particular application in the delivery of insulin, and the sensing of blood glucose levels by a glucose sensor, and thus in the treatment and/or management of diabetes, as set out above, and discussed further herein.

As noted above, the term “in temporal coordination” means that the administration of the compound is coordinated with the administration of the active agent or with the sensing of the sensor, so that the compound is present in the vicinity or still has a vasodilatory effect at the time that the administration and/or absorption of the active agent or sensing occurs. In other words, the administration of the compound is timed to coincide, or so that the effect of the compound coincides, with the administration and/or absorption of the therapeutic active agent or the time of sensing by a sensor, or is such that the compound is present in the vicinity at the time that the therapeutic active agent is administered, or while it is being absorbed, or at the time of sensing by the sensor. It will be understood that in some situations the vasodilatory effect of the compound may persist for a period of time after it has been absorbed, a phenomenon which is known to occur with hormones. It is accordingly required that the compound, or its effect, is present in the vicinity, and not necessarily that the compound itself is present; its effect may persist or be maintained after it has been absorbed. In other words, the effect of the compound may be long-lasting, and may be observed after the compound itself is no longer present. Thus, the vasodilatory effect of the compound, to increase local blood flow, occurs around the time that the active agent is being administered or absorbed, or the sensing is taking place. As discussed in more detail below, this may be achieved in various ways, including by the timings of the administration(s) and/or sensing, e.g. so that there is an administration at or around the time of administration of the active therapeutic agent or the sensing, or by prolonged or continuous or near-continuous administration, or by repeated or multiple administrations of the compound to create and maintain a high local concentration of the compound in the vicinity, such that the compound, or at least the vasodilatory effect of the compound, is present at the time that the administration of the active agent or the sensing takes place.

The term “compound with glucagon activity” includes any compound which acts at the glucagon receptor, or in other words which interacts with the glucagon receptor to stimulate the effects of the receptor. That is, a compound with glucagon activity causes or results in any of the downstream effects which arise from interaction with the glucagon receptor by glucagon. In particular, the compound interacts with the glucagon receptor and results in the dilation of blood vessels. Thus, in particular, the compound has a vasodilatory effect and more particularly it is able to have substantially the same vasodilatory effect as glucagon. The vasodilatory effect induced by the compound may be the direct or indirect result of interaction with the glucagon receptor. This includes any compound that exerts glucagon effects. Accordingly, a compound with glucagon activity may alternatively be defined as a compound which has or mimics the effect of glucagon at the glucagon receptor. A compound with glucagon activity may thus be defined as a glucagon agonist. Further, a compound with glucagon activity may alternatively be defined as glucagon or an analogue thereof.

The term “glucagon” includes any known or reported wild-type or native glucagon molecule, in any species, and any naturally occurring variants of fragments thereof. A glucagon analogue is any compound which is not a naturally occurring glucagon compound, but which has glucagon activity, or exerts glucagon effects. This includes synthetic or artificial derivatives or variants or fragments of native glucagon molecules. Glucagon analogues are known and described in the literature. Glucagon is a peptide hormone, and various glucagon analogues in the form of peptide derivatives or other peptide compounds have been developed. Thus, in one embodiment the compound may be defined as a glucagon peptide, a term which includes native or wild-type glucagon or a derivative or variant or fragment thereof which retains glucagon activity, or a glucagon analogue which is a peptide or is peptide-based. More particularly, a glucagon analogue may include one or more amino acid substitutions, additions and/or deletions compared to a native glucagon peptide, including insertions and C- and/or N-terminal truncations or extensions, as well as chemical modifications to one or more amino acid residues, including covalent modifications, such as the addition of various chemical groups (e.g. amides, esters, alkyl or acyl groups, lipophilic groups etc.). However, glucagon analogues are not limited to peptides and include any compound with glucagon activity, for example small molecule compounds.

A compound with glucagon activity includes pharmaceutically-acceptable salts of the compound, such as acid addition salts, metal salts, ammonium and alkylated ammonium salts.

Glucagon-like peptide 1 (GLP-1) is able to bind to the glucagon receptor and exert glucagon effects. Accordingly, GLP-1 is included as a compound with glucagon activity. Further, a compound with glucagon activity, including glucagon itself may act at the GLP-1 receptor. Thus, in an embodiment, also included under the general heading of “compounds with glucagon activity” are compounds which are able to interact with the GLP-1 receptor to cause a vasodilatory effect.

However, in another embodiment, the term “compound with glucagon activity” does not include, GLP, notably it does not include GLP-1, or any compound with activity at, or able to bind to, the GLP-1 receptor.

Several patent applications disclosing different glucagon-based analogues and GLP-1/glucagon receptor co-agonists are known in the art, such as e.g. WO2008/086086, WO2008/101017, WO2007/056362, WO2008/152403 and W096/29342. Other glucagon analogues disclosed are PEGylated (e.g. WO2007/056362) or acylated in specific positions of native human glucagon (e.g. W096/29342). Glucagon peptides for prevention of hypoglycaemia have been disclosed, as e.g. in U.S. Pat. No. 7,314,859.

Long-acting or more stable glucagon analogues are described in WO 2013/040678 of Novo Nordisk A/S. This document and the others mentioned above are incorporated herein by reference. Any of the compounds described in these documents may be used.

Reference may also be made to the glucagon analogue Dasiglucagon, available from Zealand Pharma A/S, and to the stabilised glucagon delivery products (pen and syringe) sold by Xeris Pharmaceuticals Inc., which may be used.

The sequence of human glucagon (glucagon 1-29) is set out in SEQ ID NO. 1 as shown below:

His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr (SEQ ID NO. 1)

Included as compounds herein are glucagon 1-30, glucagon 1-31 and glucagon 1-32, which have 1, 2, and 3 amino acid extensions respectively at the C-terminal end of SEQ ID NO. 1.

A glucagon analogue may include a peptide comprising an amino acid sequence having at least 80% sequence identity to the sequence of SEQ ID NO. 1, e.g. at least 85, 90 or 95% sequence identity.

Further, derivatives or analogues of glucagon may be made to improve the administrability of the compound. Glucagon is water-soluble, and it may in some instances be desirable to promote the fat-solubility of the compound, for example to aid in absorption of the compound through the skin. Thus, for example, more fat-soluble derivatives may be made, e.g. by attaching lipophilic groups or other fat-soluble groups to the compound.

Glucagon is known mainly for its role in the maintenance of blood glucose level, as it stimulates glycogenolysis, and glyconeogenesis from pyruvate, lactate, glycerol and some amino acids, thereby opposing the effects of insulin. Various extra-hepatic effects of glucagon have been described, such a positive inotropic and chronotropic effects, while in the gastro-intestinal tract it acts as a smooth muscle relaxant, but it also affects the glomerular filtration rate, adipose tissue, thyroid gland and central nervous system. Glucagon exerts these effects via the G-protein coupled glucagon receptor, through activation of adenylyl cyclase, increasing cAMP levels, as well as activating the phospholipase C (PLC) protein kinase C (PKC) pathway. However, besides the activation of the CAMP-dependent protein kinase A (PKA), glucagon has also been shown to activate the extracellular signal-regulated protein kinase 1/2 (ERK 1/2) in a clonal cell line of human embryonic kidney cells, as described in Jiang et al., PNAS USA, 2001, 98, 10102-10107. Any of these activities can be used as the basis of an assay to determine whether a compound has glucagon activity, or to determine the level of that activity. For example, such an assay may comprise determining whether the compound is able to increase CAMP levels in cells expressing a glucagon receptor and a membrane-bound cAMP biosensor. An assay based on cAMP detection is described in WO 2103/041678 as follows (Assay I). The assay uses HEK-293 cells having a membrane bound cAMP biosensor (ACTOne™) into which a glucagon receptor is cloned. The cells (14000 per well) are incubated (37° C., 5% C02) overnight in 384-well plates. The next day the cells are loaded with a calcium responsive dye that only distributes into the cytoplasm. Probenecid, an inhibitor of the organic anion transporter, is added to prevent the dye from leaving the cell. A PDE inhibitor is added to prevent formatted CAMP from being degraded. The plates are placed into a FLIPRTETRA and the compound to be tested for glucagon activity is added. End-point data can be collected after 6 minutes. An increase in intracellular CAMP is proportional to an increased in calcium concentrations in the cytoplasm. When calcium is bound a fluorescence signal is generated. EC50-values may be calculated in Prism5.

By way of example, a compound with glucagon activity may be any compound, e.g. a glucagon peptide, that binds to a glucagon receptor, or activates it, with an affinity or potency (EC50) below 1 μM, e.g. below 100 nM or below 1 nM, for example as determined by a CAMP assay as described above.

The term “insulin” as used herein includes insulin molecules of any animal species, particularly human, and analogues and derivatives thereof, including artificial and synthetic analogues. Various analogues and derivatives of insulin are known and reported in the art and in clinical use today. Any such insulin compound is included. Insulin analogues and derivatives include compounds and peptides having sequence-modified amino acid sequences and/or chemical modifications analogous to those described for glucagon above.

Fast-acting insulin analogues are available. Fast-acting analogues are readily absorbed from a sub-cutaneous injection site, and may act faster than natural insulin. Such analogues may be useful to supply the bolus level of insulin needed at mealtime (prandial insulin). Examples of such analogues include Lispro, Aspart, and Glulisine. Long-acting insulin analogues are also available, but such analogues would not typically be used according to the disclosure herein. Thus, in particular, the insulin analogues and derivatives herein are those with an activity profile similar or comparable to that of a native insulin, those which are fast-acting, those which are used with meals, those which are used in insulin pumps, and in particular those used in artificial pancreases.

The term “diabetes” includes all types and forms of diabetes mellitus, including type 1 (T1D) and type 2 (T2D). Whilst the uses, methods and systems herein have particular utility in the treatment or management of T1D, glucose monitoring, particularly continuous glucose monitoring (CGM) may be needed in all types of diabetes, and the administration of insulin may be needed in certain subjects with T2D, for example those with prolonged and/or advanced disease, where insulin production may be reduced. The term “diabetes” also includes any diabetic state, or indeed any state or condition where external control of glucose levels may be needed or may be of clinical benefit. This includes conditions where the pancreas has been damaged or removed, or is not fully functional to produce insulin, for any reason, for example as the result of disease or trauma. In an embodiment, the diabetes which is treated or managed as described herein does not include T2D.

A “therapeutic active agent” may alternatively be referred to a drug, and includes any agent, e.g. any compound, substance or moiety, which exerts a beneficial or therapeutic effect on the subject to which whom or to which it is administered. It is thus a pharmaceutically active agent (e.g., a pharmaceutical compound), and includes any agent with clinical utility. As noted above, insulin is a particular therapeutic active agent for use herein, but the active agent may be any agent known, reported or proposed for medical use, to treat or prevent any medical condition or disease.

The therapeutically active agent may be administered to the subject in an amount which is effective for the agent to exert or achieve its intended therapeutic effect. For example, this may be to cure, alleviate, arrest, delay the progression of, or in any way improve the condition to be treated, or any symptom thereof.

In one embodiment, the condition to be treated is any condition responsive to, or which benefits from, the therapeutic active agent. In another embodiment the condition does not include T2D.

Likewise, the compound with glucagon activity may be administered in an amount which is effective to achieve a local vasodilatory effect, or an effect of increasing local blood flow at the site of its administration. Vasodilatory activity may be assessed, or determined, by measuring blood flow by a laser Doppler method, at an administration site on or in a human or non-human animal subject after administration of the compound. For example, the compound may be injected SC and blood flow just below the skin surface may be determined at the injection site. Such a method is described in Example 1. However, for the methods and uses herein where the compound is used for its effect in the delivery of a therapeutically active agent (e.g. insulin) or in conjunction with the operation of a sensor (e.g. glucose sensor), it is not necessary, and indeed it may be undesirable, for the compound to exert its usual therapeutic effect of increasing blood glucose, or counteracting an episode of hypoglycaemia. The amount, or dose, administered may thus be less, and indeed typically will be less, than a dose which is administered, or typically used, to treat hypoglycaemia. Doses for the compound are discussed in more detail below.

The terms “treatment” and “treating” and other variants thereof as used herein refer to the management and care of a subject for the purpose of combating a condition (which as indicated above, includes any disease or disorder). “Prevention” includes preventing or delaying the onset of the condition, or any symptom, manifestation, or complication thereof. The therapeutically active agent may be administered to treat or prevent any condition which is responsive to, or which benefits from, the administration of that agent.

The subject may be any human or non-human animal subject, particularly mammalian subject, more particularly a human subject. The methods, uses, and systems presented herein find particular utility in the treatment or management of human subjects with diabetes. However, veterinary uses also included, and the subject may be any livestock, domestic, sports, zoo, or laboratory or research or wild animal. Accordingly, the subject may for example be a canine, feline, equine, bovine, ovine or murine animal etc.

A “sensor” as referred to herein is a device for determining the level of an analyte. The analyte to be determined is referred to as a target analyte. The sensor herein is a bodily sensor. That is, it is a sensor which is worn or carried in or on the body of the subject. The device may thus be in contact with an internal and/or external body surface of the subject, e.g. a tissue of the subject. It may in effect be worn on or within a tissue of the subject. The device is able to determine the target analyte directly in the body tissue or fluid with which a sensing element of the sensor device is in contact (e.g. a sensor probe, for example a sensor electrode), or to sample a body fluid or tissue of the subject and detect and determine the amount of the target analyte present in the sample. For example, the device may be able to measure the target analyte directly in the interstitial fluid of any body tissue. This may be achieved by a sensor element (e.g. probe) provided in the device which comes into contact with, or is positioned in the tissue. In a representative example, the sensor may be a sub-cutaneous (SC) sensor, but it may be designed to be located at other sites or surfaces in or on the body, for example intraperitoneally (IP) or in other body cavities or organs. The sensor may thus be an external, a partially in-dwelling device, or a fully in-dwelling device.

Typically, a sensor and/or delivery device, e.g. an artificial pancreas, is partially in-dwelling, where the controller (control system), power, and pump elements are external, and are connected to the subject through delivery lines (e.g.

infusion tubes) which are internal in the subject. Tubing-free (“patch pump”) versions are also available. However, fully in-dwelling devices are not excluded. Thus, whilst partially in-dwelling devices may be powered by external battery packs and/or be provided with external control systems, fully in-dwelling device with internal (in-dwelling) batteries and control systems, and for example means for wireless communication may be provided. Advances in anti-fouling technology and battery capacity mean that fully in-dwelling devices may be implanted for prolonged periods of, for example, 3, 4, 5, 6, 7, 8, 9, 10, 11, or 12 months or more. Furthermore, fully in-dwelling devices may be charged from external power sources at the skin over the in-dwelling device.

In line with this, the term “sensing” refers generally to the determination of an analyte, or more particularly the level of an analyte, in the subject. “Sensing” may be viewed as the taking of a reading, or determination, of the analyte at a time point. Of particular concern in the uses, methods and systems herein is the determination of the blood level of an analyte, that is the level of an analyte that is present in the blood of the subject. This does not mean that the sensor is required to perform the determination directly in or on the blood of the subject, although this is not precluded, but that it is able to provide information that is indicative of the level of the analyte in the blood of the subject. For example, the sensor may measure the level of the analyte in the interstitial fluid, which a SC sensor for example is able to access, and from that to estimate the level of the analyte that is present in the blood. Accordingly, there may be a predetermined relationship between the level of that analyte in the body tissue or fluid that is sampled and the level of the analyte in the blood, or such a relationship may be determined. The relationship may be used to determine the level of the analyte in the blood. In other words, the value determined for the level of that analyte in the sampled body tissue or fluid may be converted to a value for, or indicative of, the level of the analyte in the blood. This may be done by reference or comparison to reference charts or graphs, or calibration curves etc., according to principles known in the art, or may be performed by software or algorithms, e.g. by a control system of a device which implements the medical uses and methods set out above. “Determination” thus includes direct and indirect measurement, or estimation or assessment, of the level of the analyte in the blood. The concentration of the analyte may be determined, or any other measure or value indicative of the level of the analyte.

Such a sensor provides a subject with the convenience of being able to determine, or monitor or track, analyte levels themselves. It is to be understood, however, that this does not preclude that the sensor may also be used partly or exclusively by clinicians or other medical staff. A bodily sensor further does not require a sample to be separately taken and administered or applied to the sensor. A bodily sensor automatically takes a sample or reading when needed or when programmed or instructed to do so. A variety of such sensors are known and available in the art, as typified by glucose sensors which are routinely used by diabetic subjects in the management of their diabetes.

The analyte may be any analyte it is desired to determine, notably in the blood of a subject. Glucose is a typical representative analyte, but it may be any other molecule which occurs in the blood of a subject. For example, it may be a metabolite such a pyruvate, or lactate, e.g. which is indicative of a state or condition of a subject. It may for example be desirable to track or monitor such analytes, for example to monitor energy expenditure or utilisation, including during and after surgery or hospitalisation, fitness or recovery from sports etc. Such monitoring of analytes may be of benefit in monitoring patients in intensive care units.

For determination and monitoring of glucose levels so called continuous glucose monitoring (CGM) sensors have been developed. The sensor may accordingly be a CGM sensor. The term “continuous” here does not imply that the sensor is continually sensing glucose without interruption, but rather that repeated determinations (sensing) occur over a period of time, for example over a period of 24 hours, or longer. The “continuous” sensing may occur over the time period the sensor is on or in the body. Sensing may occur at regular intervals, and/or at pre-determined or programmed intervals, e.g. at fixed intervals. For example, sensing may take place at 5 minute intervals, or at longer intervals. Alternatively or additionally, the sensing may occur, or may take place with greater frequency at times when blood glucose levels are changing, or it is predicted that they will be changing. For example, there may be an increased frequency of sensing at meal times, or times of activity, and/or a reduced frequency of sensing at night. The user may inform or instruct the sensor when to take readings, or this may occur automatically, or both.

One or more sensors may be used at a subject at any one time. For example, sensors may be positioned at different sites or locations in or on the body. This may be useful to introduce redundancy, for example in the event that one sensor fails, another sensor will be able to take readings. Further, it can be advantageous to have readings taken at different sites or different tissues, and/or to use different sensor modalities at different sites. In this way a robust system can be provided. An artificial pancreas or a glucose sensor system may contain one or more glucose sensors. The compound with glucagon activity may accordingly be administered in conjunction with one or more sensors.

In an embodiment, the compound is administered each time sensing by the sensor occurs, i.e. each time a sensor takes a reading. However, this is not necessary as the compound can be administered such that it is present or exerting an effect in the vicinity of the sensor when it is sensing. For example, the duration of action of the compound may persist over a period of time (e.g. 20, 30, 40, 50, or 60 minutes or more), during which time more than one reading may take place. Alternatively, the compound may be administered continuously or over a prolonged period of time, for example from a controlled release preparation (e.g. a slow-release reservoir) or by continuous infusion, such that it is present in the vicinity of the sensor each time sensing by the sensor occurs. In an embodiment there may be controlled release over a period of time during which one or more sensor readings may take place. Accordingly, the compound may be administered in a manner, for example often enough, to have a more or less continuous effect on local blood flow, and thus on sensor performance. This can range from every time a reading takes place to a few times a day.

The compound with glucagon activity is administered to a site in the vicinity of the site of administration of the therapeutic active agent (e.g. insulin), or the site of the sensor, more particularly the site at which the sensing or sampling by the sensor occurs. The vicinity of a site may be defined as a region within 3 cm from the site, more particularly a region within a radius of 3 cm from the site. In some embodiments, the vicinity may be a region within 2.5, 2, 1.5 or 1 cm of the site, or more particularly with a radius of 2.5, 2, 1.5 or 1 cm from the site.

In a particular embodiment, “in the vicinity” may be defined as within 3 cm, or within 2.5, 2.0, 1.5, 1.4, 1.3, 1.2, 1.1 or 1.0 cm of the sensor, or more particularly still, of the sensing or sampling element of the sensor device (e.g. the sensor probe, e.g. electrode or equivalent element).

In another embodiment, the reference to “in the vicinity” is made with reference to the centre of the sensor, or the centre of the probe of the sensor. That is, in this embodiment, “in the vicinity” means within 3 cm, or within 2.5, 2.0, 1.5, 1.4, 1.3, 1.2, 1.1 or 1.0 cm of the sensor, or more particularly still, of the sensing or sampling element of the sensor device (e.g. the centre of the sensor probe, e.g. electrode or equivalent element).

In particular embodiments, the compound may be administered in close proximity to the site of administration of the therapeutic active agent (e.g. insulin), or the site of the sensor, more particularly the site at which the sensing or sampling by the sensor occurs (wherein this site is defined according to any embodiment above). “In close proximity” means a region within 2 cm from the site, or more particularly, a region within a radius of 2 cm from the site, e.g. 1.5 or 1 cm. As noted above, in the context of a sensor, this may be of the sensor itself, or more particularly of the sensing or sampling element of the sensor device, or the centre thereof.

The compound is administered in temporal coordination with the administration of the therapeutic active agent to be delivered (e.g. insulin) or with the sensing by the sensor. This means that the administration of the compound is timed to coincide with the administration of the therapeutic active agent or the time of sensing by a sensor, or is such that the compound is present, or is active or still has an effect, in the vicinity at the time that the therapeutic active agent is administered and/or is being absorbed, or at the time of sensing by the sensor. Thus, the vasodilatory effect of the compound, to increase local blood flow, occurs around the time that the active agent is being administered or the sensing is taking place. In this way the administration of the agent, or its delivery, or the sensing by the sensor benefits from the increased local blood flow, as discussed in more detail below. In other words, the compound is administered in synchronicity or coincidentally with the administration of the active agent, and/or the sensing, or it is administered such that the compound is present or is effective in the vicinity of the administration site of the active agent and/or the sensing in synchronicity or coincidentally with the time or administration and/or absorption and/or sensing. Put another way, the administration of the compound is synchronised or coincident with the administration of the active agent and/or sensing by a sensor, or the administration of the compound delivers the compound synchronously or coincidentally with the administration and/or absorption of the active agent and/or sensing by a sensor.

Effectively, the compound is administered, or is administered or delivered to be present and/or active or effective, at the same time, or substantially the same time (i.e. about the same time) as the active agent, or the sensing. Thus, the compound may be administered before, during, or shortly after the administration of the therapeutic active agent, or the sensing. For example, the administration may be within 30 minutes, or more particularly within 25, 20, 15, 12 or 10 minutes, or 6, 5, 4, 3, 2 or 1 minute(s) of the administration of the therapeutic active agent or the sensing. As can be seen from FIG. 15, the vasodilatory effect of the glucagon can be seen over a period of time of some minutes (35-40 minutes or so), and further the vasodilatory effect remains above baseline for a period of time. Thus, the vasodilatory effect of the compound may persist for some hours after administration. Further, an active agent such as insulin may be absorbed for an extended period of time, e.g. 2-3 hours after it is administered, and therefore there is not a need for an exact coordination of administration time of the compound and the active agent. Accordingly, there is some latitude in the timing of the administrations and sensing, and it does not have to be at exactly or precisely the same time. Indeed, as discussed above, the key issue is that the compound is present or can have a vasodilatory effect in the vicinity of the therapeutic active agent administration site or the sensor sensing site at the time that the administration and/or absorption or sensing takes place. This can be achieved using repeated or prolonged administrations of the compound. This may create an increased local pressure in the tissue that may counteract diffusion effects. Accordingly, the administration window may be longer, for example 35, 40, 45, 50, 55, 60 minutes, or more, as long as the administration is such as to ensure the presence of the compound or vasodilatory effect of the compound in the vicinity at the time of administration and/or absorption of the active agent, or the sensing. This may be achieved by repeated multiple administrations over a period of time, e.g. the course of a day, or by continuous administration, e.g. continuous infusion or controlled release from a slow release preparation.

The timing may be determined by the nature and mode of the administration, and the formulations (compositions) that are administered. For example, the compound may be co-formulated with the active therapeutic agent in the same composition (i.e. they may be provided or used in admixture), in which case it will be seen that the administration is simultaneous. In another example, the active agent and the compound may be provided in separate formulations, but they may be mixed in use, just prior to administration, for example by mixing prior to injection manually or within a delivery device (e.g. in an artificial pancreas, or a delivery system). For example, they may be mixed or administered in the same delivery line, or two separate delivery lines may become joined prior to the point of administration etc. Alternatively, in another example, the compound and the active agent may be administered separately, for example in separate injections or delivery lines. In yet another example, the compound may be delivered over a period of time during which the therapeutic active agent is administered or the sensing takes place, e.g. from a slow or controlled release preparation, or by other continuous administration, or repeated administrations. Such different modes of administration are shown in FIGS. 7, 8 and 9, for example. Typically, where the administration is separate, the compound may be administered before the active agent. In the case of a sensor, the administration may be timed to take place before or simultaneously with the time of sensing by the sensor, for example within a few minutes in advance, e.g. 6, 5, 4, 3, 2 or 1 minute(s). However, as discussed above, due to the duration of action of the administered compound, the time of administration prior to sensing can be longer, e.g. 30, 25, 20, 15, 12, or 10 minutes etc.

Thus the administrations and the sensing may be simultaneous, or sequential, as long as they are temporally coordinated as discussed above. In the case of administration of the active agent, this may be the same administration, or a separate administration. Details of different administration routes etc. are discussed below.

As noted above, the effect of the compound is to improve local blood flow, or more particularly to increase local blood flow at and around the site of its administration. The local blood flow may be increased in the vicinity of its site of administration, “vicinity” being defined as above. The increased blood flow has the effect of improving the effect of the therapeutic agent which is co-administered with the compound, or improving sensing by the sensor. For example, the effect of insulin may be improved, or the performance of the glucose sensor may be improved, that is the determination of blood glucose level by the glucose sensor may be improved.

By improved effect is meant the therapeutic benefit or effect that the therapeutic agent is administered to achieve is improved. This can be in any way, for example a faster and/or larger effect is achieved, or a lower dose of the active agent is enabled, pharmacokinetics and/or pharmacodynamics are improved etc. In the case of insulin, this effect may be defined as improved glucose control. Thus, the effect may be the effect of reducing the level of glucose in the blood. An improved effect of insulin may accordingly be a faster, or more rapid, decrease of blood glucose level, or a faster onset of a reduction in blood glucose level. For example, there may be a reduced delay in the time of response of the subject to the insulin (or indeed other therapeutic agent), or more particularly the response of the blood glucose level, or a reduced lag or latency in the response to the insulin. In other words, the pharmacodynamics of the response to the insulin may be improved, for example faster. The effect of insulin in stabilising or normalising blood glucose level may be improved, for example by achieving a faster effect or faster response of the blood glucose level to the insulin. It may also lead to a more predictable absorption of insulin and thereby also a more predictable effect on glucose levels by reducing the day to day variation in insulin absorption observed with SC insulin injections. These various effects may be seen relative to, or compared to, the effects achieved with the same insulin administration in the absence of the compound. Another aspect is that the time that one dose of insulin works on glucose levels may be decreased reducing the risk of hypoglycemia. Similar or analogous considerations may apply to other therapeutic agents.

Similarly, in the case of a sensor, the dynamics of the sensing may be improved, or faster. This may be evident in faster sensing, for example a quicker result, or more particularly a reduction in a delay, lag or latency in the sensing, and/or in a more accurate result. Such an improvement may be seen relative to, or in comparison with, the sensing obtained in the absence of the compound, i.e. relative or compared to the effect (or result) achieved by the sensor in the absence of the compound.

Without wishing to be bound by theory, the effect of the compound, or of the improved local blood flow, may be to increase absorption of a co-administered therapeutic active agent, e.g. insulin.

Increased absorption may be seen as an aspect of improved delivery of the administered therapeutic active agent. That is, the delivery of the active agent to its target tissue, or to the blood stream (circulation) may be improved, for example increased or speeded up (in other words the amount of therapeutic agent delivered or the rate of delivery may be increased). Thus, more generally, the compound may have the effect of improving delivery of a co-administered therapeutic active agent to a subject. Delivery in this context can be taken to refer to the delivery of the therapeutic active agent to its site of action or uptake in the body, e.g. the delivery of the agent (e.g. insulin) to a site where it can absorbed by the body, including into the circulation.

The compound with glucagon activity is administered to the vicinity of the administration site of the therapeutic agent or the sensor to achieve a vasodilatory effect, rather than a glucose-increasing effect. The dose of the compound may be selected or determined accordingly. It has surprisingly been found that the dose required to achieve a vasodilatory effect is small, much smaller than has been reported to be used for the current therapeutic or diagnostic indications for glucagon.

In certain body regions a vasodilatory effect has been observed in some subjects with doses <0.01 mg of glucagon.

Generally, therefore, a small dose is used, and typically a smaller dose than would be administered in order to treat or to counteract hypoglycaemia, or an episode of hypoglycaemia. The dose is thus smaller than a rescue dose administered in the case of hypoglycaemia. Such a rescue dose is typically 1 mg.

In particular, the compound may be administered to the subject at a micro-dose which is less than the dose required to counteract actual hypoglycaemia, or less than the dose required to achieve a clinically significant increase in the level of glucose in the blood. A very small dose, or micro-dose, of a compound with glucagon activity although not intended to increase blood glucose or to treat hypoglycaemia, may nonetheless have a detectable or measurable effect in increasing blood glucose level. The term “clinically significant” is meant to convey a dose which is sufficient to achieve an increase in blood glucose level which is of benefit in treating hypoglycaemia. Hypoglycaemia is defined as a blood glucose level of <3.9 mmol/l (<70 mg/dl). More particularly the hypoglycaemia may involve a blood glucose level of <3.9 mmol/l coupled with clinical signs that the affected subject is in need of assistance (e.g. is in need of clinical assistance).

In an embodiment, the compound is administered at a micro-dose which is no more than, or less than 0.2 mg, more particularly no more than, or less than 0.15 mg. In a more particular embodiment the compound is administered at a micro-dose which is no more than 0.1 mg, or no more than 0.09. 0.08, 0.07, 0.06 or 0.05 mg.

In the context of the treatment or management of diabetes, a compound with glucagon activity may separately be administered to treat or prevent hypoglycaemia. That is, the compound may further or additionally be used for its conventional or hormonal purpose, to increase the level of glucose in the blood. It will be understood that since glucagon has opposing effects to insulin, such use of the compound will involve administering the compound at an entirely separate, and different time to the insulin. In other words, such a use of the compound for its therapeutic glucose-increasing effect is not in temporal coordination with insulin administration. In such use the compound is administered to raise glucose, whereas insulin is administered to lower glucose in the blood, and hence they would not be administered at or around the same time, but rather at opposing times. A dose of the compound administered for such an effect is referred to herein as a therapeutic dose (in contradistinction to the “enhancing” dose administered to improve the effect or delivery of the therapeutic agent, or the functioning of the sensor).

Whilst conventionally a dose of compound administered to treat or counteract actual clinical hypoglycaemia, i.e. a rescue dose, is large (e.g. of the order of 1 mg or so, or 0.5 mg in children), smaller doses of the compound may be used, according to the disclosure herein, in the management of diabetes, or when it is detected, e.g. by a sensor or by clinical signs, that blood glucose levels are starting to drop. Further still, small doses of a compound with glucagon activity may be administered periodically, or at particular time intervals, or at fixed or predetermined points of time, e.g. at times of predicted hypoglycaemia, in order to prevent hypoglycaemia from occurring, or to reduce the chance or risk that hypoglycaemia occurs, or to reduce the extent of the hypoglycaemia. Such prophylactic doses, or “management” doses, may be in the order of the micro-doses indicated above. Repeated small or micro-doses of the compound may be sufficient to prevent hypoglycaemia from occurring.

For such therapeutic use, the therapeutic doses of the compound may be administered by the same means as the “enhancing” dose, for example in the context of a delivery device or delivery system (e.g. AP), from the same reservoir and by the same line or channel etc. Thus, the site of the administration of the compound for the two different purposes may be the same, although it may alternatively be different. However, the therapeutic administration of the compound to counteract hypoglycaemia will be temporally spaced apart, and distinguished from the administration of the compound in order to achieve a vasodilatory effect in conjunction with the further therapeutic active agent (insulin) or sensing. In an embodiment administration for a therapeutic effect to counteract hypoglycaemia will be at a completely different time to administration for the enhancing effect on insulin. In an embodiment, there may be prolonged or controlled release of the compound for its vasodilatory effect (i.e. in conjunction with the delivery of the therapeutic agent and/or analyte sensing) and a separate administration for the effect of counteracting hypoglycaemia. Generally, such administrations may be at different doses or different dosage rates.

In another embodiment, administration of the compound for its therapeutic anti-hypoglycaemia affect may be the same or similar doses and/or administration routes as the administration for the vasodilatory (enhancing) effect. In this case, in an embodiment the two administrations may be distinguished by site of administration, i.e. they may be at different sites. In particular, in an embodiment, the therapeutic administration will not be in the vicinity of the site of administration of the therapeutic agent or the sensing by the sensor.

In terms of the vasodilatory effect (or alternatively put, the “enhancing” effect), the methods, uses and systems herein involve administering the compound with glucagon activity for one of two purposes or both, namely in conjunction with administration of an active therapeutic agent or with a sensor. These two uses may be independent of one another, or they may be carried out together. Thus, the sensing of the analyte by the sensor may complement, and indeed may inform, the administration of the therapeutic active agent. Accordingly, the administration of the active agent with the compound may occur in response to the sensing by the sensor. This may particularly be the case in the context of insulin administration and glucose sensing. However, it may also be the case that the therapeutic active agent is co-administered with the compound in response to sensing by another sensor, or another result, e.g. a different sensor, for example a sensor which is not a bodily sensor, or which is not used in conjunction with the compound.

The compound with glucagon activity and the further therapeutic agent (e.g. insulin) will typically be provided for administration in the form of a pharmaceutical composition whether combined in a single composition, or more suitably as separate compositions. A pharmaceutical composition may comprise the compound or agent and one or more pharmaceutically acceptable carriers or excipients.

Such carriers or excipients are well known and described in the pharmaceutical art, and will depend on the route of administration of the composition. The compound (or composition) may be administered by any desirable route, and this may depend on the nature of the therapeutic agent.

Typically, to take advantage of the vasodilatory effect of the compound, the compound and the therapeutic agent will be administered to a selected or desired site or location in the body where the absorption can be enhanced by local vasodilation. A particular utility arises in this regard in tissues with low perfusion, where it can be difficult to achieve sufficient local concentration of a drug to have the desired effect. This can sometimes be the case with antibiotics. The present uses, methods and devices may thus find application in the administration of antibiotics for local administration of an antibiotic for treatment of localised infections.

Thus, the administration may suitably be by injection or infusion to a body site. The administration may be topical to the site of a body tissue or organ, or to a body cavity. In an embodiment, the administration is to a site which involves absorption, e.g. in the skin, under the skin, in a muscle, or in the abdomen, in the nose, or at a mucosal surface, for example in the respiratory tract, e.g. in the lung. For example, the administration may be sub-cutaneous (SC), or intra-muscular (IM), or intra-peritoneal (IP), or to any desired body cavity or organ.

In some embodiments the administration may be to the oral cavity, e.g. buccal or sublingual, or nasal (e.g. by nasal spray), pulmonary (e.g. by inhalation), vaginal, rectal, ocular, or uretral.

In an embodiment the administration of the compound is not pulmonary, e.g. not by inhalation. In another embodiment, where the compound is GLP-1, the administration is not pulmonary or not by inhalation. In a still more particular embodiment, where the condition to be treated is T2D and/or where the sensing is glucose sensing in a subject with T2D, the administration of the compound is not pulmonary or by inhalation.

Particularly, the administration may be parenteral.

In particular embodiments the administration is SC or IP.

Similarly, the sensor may be located at any desired site or location in the body. This may include any site, tissue or organ where there is a rich blood supply, including for example the nasal cavity (as this reduces sensor delay). However, typically the sensing will be SC or IP. A SC sensor may be worn externally on the body, and perform SC sensing, e.g. the sensor may access the body fluid or tissue SC. A SC sensor may be positioned at any desirable or convenient site on the body, and this may depend upon whether it is a stand-alone sensor, or part of an integrated device. A typical location for a glucose sensor is on the abdomen, but the sensor may for example also be positioned on the arm, e.g. upper arm, or leg, e.g. thigh. Alternatively, the sensor may be partially in-dwelling, and may for example perform sensing at an internal body cavity, e.g. IP or at a body tissue or organ. This may include for example sinuses, or to determine an analyte in the CSF, a sensor may be positioned in the skull or along the spinal cord. There may be value for example, in sensors to determine metabolites at such sites, for example in diagnosing or monitoring metabolic diseases or the treatment thereof, or in therapeutic monitoring more generally.

The pharmaceutically acceptable carriers or excipients may comprise a buffer system, preservative(s), tonicity agent(s), chelating agent(s), stabilizer(s) and surfactant(s). In one embodiment of the invention the pharmaceutical formulation is an aqueous formulation, i.e. formulation comprising water. A range of such agents and components are known in the art and available to the skilled practitioner.

Further possible additional ingredients may include wetting agents, emulsifiers, antioxidants, bulking agents, metal ions, oleaginous vehicles, proteins (e.g., human or non-human serum albumin, gelatin, or other proteins) and a zwitterion (e.g., an amino acid such as betaine, taurine, arginine, glycine, lysine and histidine). Other ingredients may include carriers such as for example polymers, particles, encapsulating agents and such like.

As well as formulation aids, the pharmaceutical composition may also contain other ingredients or components, including for example, agents which may assist in the administration or delivery of the compound, for example, penetration-enhancing agents, for example, skin penetration-enhancing agents, depending on the mode and site of administration, for example where the compound is applied to the skin in the vicinity of a SC sensor or SC administration of the therapeutic agent. Such skin penetration enhancers are widely used in the cosmetic and pharmaceutical fields to promote the penetration of drugs or other agents through the skin. They may work in different ways, directly on components in the skin, or indirectly. Typical such agents include azones, urea, fatty acids, sulphoxides (e.g. DMSO), surfactants, terpenes, alcohols, e.g. ethanol and glycols. They may alternatively be vesicular carriers (including e.g. liposome or other microvesicles), or enzyme inhibitors which alter the lipids in the stratum corneum.

In some embodiments, it may be convenient to administer the compound in a prolonged, or slow, release format (also referred to herein as “controlled release”). For example, at the site of a sensor, it may be administered in a slow release formulation or preparation, or a slow release reservoir. In the case of SC sensor, this may take the form of a cutaneous adhesive patch which is positioned or applied in proximity to the sensing site. This is convenient as a SC sensor is fixed to the skin. A transdermal delivery device for glucagon, e.g. an adhesive patch or reservoir for transcutaneous delivery, may be provided as part of the sensor, for example as shown in FIG. 5, or as a separate patch or reservoir for application to the skin in the vicinity of the sensor. In the case of a SC delivery device, e.g. infusion pump, for a therapeutic active agent (e.g. insulin), the SC delivery line may also be fixed to the skin, and so again the use of a cutaneous patch or reservoir for transcutaneous delivery for slow release administration of the compound is convenient, in proximity to the site at which the delivery line enters the skin, for example as shown in FIG. 9 or 11. In such a format, it may be beneficial to use a skin-penetration enhancer to facilitate or assist in the delivery or absorption of the compound, or to use a more fat-soluble derivative, as discussed above.

An alternative slow release format is to provide the compound in a slow release coating on a delivery line of a delivery device (e.g. an infusion pump), in particular at the tip, or at or towards the end of the delivery line, where it enters the skin for example, or a sub-cutaneous or other internal part of the sensor, for example the sensor membrane, a sensor needle or electrode (such as is present in a glucose sensor for example) or the sampling part of the sensor, such that the compound is administered when the therapeutic agent is administered or when sensing by the sensor takes place.

Still another slow release format is a depot formulation (or in other words a composition), or implant, comprising the compound together with a slow release carrier or material which delays or prolongs the release of the compound. Such a preparation may be administered to, or deposited at, a site in the vicinity of the administration site of the therapeutic agent, or at the site of sensing by the sensor. Thus, such a preparation may be incorporated a part of the sensor, for example, at or around the sensor needle or electrode, as shown for example in FIG. 4. Slow release carriers and materials suitable for such use are known in the art, and include for example various polymeric materials. An example of such a slow release format is a micro-ampoule or capsule, which contains, or encloses or encapsulates a formulation (composition) of the compound in a manner which permits slow release of the compound (for example through the wall of the capsule), and which may be administered to the site of insulin delivery or sensing.

The compound with glucagon activity may be administered in different ways, depending on choice, and the design of the device by which it is administered, for example. Thus, administration by injection or infusion may be acute or prolonged over a period of time, for example acute bolus injections, which are administered over 1-3 seconds, for example, or prolonged bolus injection, for example over 10-15 seconds. Infusion may be over a longer period of time, for example minutes or hours. A continuous infusion may be performed. Thus, administration of the glucagon may be continuous or intermittent, and over a varying duration of time, ranging from seconds to minutes or hours, or it may be continuous over a period of days. For example, infusion may be stopped during the night when glucose excursions are limited, and may be intermittent during the day. This may depend on the site of administration, but in general such injections or infusions may be applicable to sub- cutaneous injection or infusion, or to injection or infusion at other sites, e.g. IP.

In the treatment or management of diabetes, insulin is typically administered by parenteral means, commonly by SC or IP injection or infusion and these represent preferred administration routes herein.

Insulin is typically administered by some subjects by multiple daily injections (MDI). Thus, in some embodiments, the compound is co-administered with the insulin in MDI. The insulin may be co-formulated with the compound, e.g. in admixture, or the insulin and compound may be mixed prior to administration, e.g. prior to take up in the syringe. Alternatively, the insulin and the compound may be administered in separate injections, e.g. in separate MDI. In this way the dose, or bolus, of the compound may be administered by separate injections throughout the day. This may be at different frequencies throughout the day.

Another common administration route for insulin is by infusion pump, commonly termed an insulin pump, i.e. continuous subcutaneous insulin infusion (CSII). The compound may be administered alongside the insulin via the insulin pump, or via a different pump. The skilled practitioner will recognise that this may be done in a variety of ways. For example, the compound may be co-formulated with the insulin for delivery via the pump, or the pump may comprise or be provided with a separate reservoir for the compound, which may be delivered by the same or different delivery line as the insulin. Two separate delivery lines may be joined into one prior to entry to the skin etc. In an embodiment, there may be a continuous infusion of the compound, for example micro-doses of the compound. In another embodiment, the administration of the compound may be timed with the insulin.

The insulin and compound may be administered from devices which administer them via microneedles through the skin. These could for example, be short, 1-3 mm microneedles, as commonly known and used in delivery devices in the art. The delivery needles for each may readily be configured to be close to one another. In an embodiment, the needles for administration of the compound and insulin may be positioned close to one another in the device, but may penetrate the skin to different depths. For example they may be of different lengths.

Further, in the management of diabetes it is commonplace for a subject to use a bodily glucose sensor, such as a CGM sensor. Typically, this is a sub-cutaneous sensor. Accordingly, for such a glucose sensor, and particularly for a SC glucose sensor system, it is preferred for the compound to be administered SC. Thus, the compound may be administered as part of an insulin delivery system, or integrated pump system for delivery of the compound and insulin.

As noted above, artificial pancreases (APs) have been developed to automate insulin delivery and glucose sensing in an integrated fashion. An AP may thus be regarded as an integrated device, or integrated system, for controlling blood glucose level, comprising one or more glucose sensors, and a control system and delivery device for insulin. According to the developments herein, the AP is modified further to comprise a delivery device for the compound. This may be the same delivery device, or a separate delivery device to the insulin delivery device. Thus, an AP represents another way of administering the compound.

A delivery device, or more generally a delivery means, may take the form of a pump, or injection or infusion device, or a transdermal delivery system, e.g. an adhesive patch or reservoir for transcutaneous delivery which is in contact with the skin-contacting part of a device etc. or as a coating, depot or reservoir in the device, which is configured to allow the compound to be delivered or released at the site of glucose sensing and/or insulin administration

Accordingly, in various embodiments, the compound and/or the insulin may each be administered:
(i) from an artificial pancreas, being an integrated device comprising one or more delivery devices for administration of the insulin and the compound, and a glucose sensor; or
(ii) from an insulin pump and/or a pump for delivery of the compound, which may be the insulin pump or a different pump; or
(iii) as part of a regime of multiple daily injections; or wherein:
(iv) the compound is administered by continuous infusion in the vicinity of the site of insulin administration or the glucose sensor; or
(v) the compound is administered in the form of a slow release preparation.

Different slow release preparations are discussed above.

APs in clinical use today tend to be SC APs. However, as noted above, IP AP are being developed combining IP insulin administration with IP glucose sensing, or mixed SC/IP APs, for example where SC glucose sensing is combined with IP insulin delivery. The present methods and uses are applicable for use with any such AP.

In certain embodiments, the compound may be used in conjunction with a SC glucose sensor, and the insulin may be administered SC, or by any means, with or without co-administration of the compound. In other words, in some embodiments the compound may be used to improve glucose sensing alone, without using it in conjunction with insulin administration. This may be a desirable option for example, when the glucose sensor is SC, and insulin is administered IP, or in another body cavity.

Accordingly, in an embodiment, the compound is administered subcutaneously and insulin is administered subcutaneously, intraperitoneally, or in any other body cavity or organ or tissue, e.g. intra-muscularly.

Further, in an embodiment, glucose sensors are subcutaneous, intraperitoneal, or placed in any other body cavity or organ or tissue.

In any such embodiment, the organ may not include the lung. More particularly, in one embodiment where the compound is GLP-1, the organ does not include the lung.

In further embodiments, the compound is administered subcutaneously in conjunction with subcutaneous glucose sensor(s). Such embodiments may comprise the further administration of insulin, for example IP, with or without co-administration of the compound.

It will therefore be understood that the use of the compound to augment delivery of a therapeutic agent, and the use of the compound to augment a sensor may be employed independently of one another, or in combination.

In any such embodiments, the compound may further be separately administered subcutaneously to increase the level of blood glucose when required. This further, separate, administration of the compound to counteract or prevent hypoglycaemia may be at the same site as administration of the compound in the vicinity of the insulin administration site or the glucose sensor site, or may be a different site.

In certain embodiments as discussed further below, the methods and uses herein may conveniently be applied in various automated formats, including a stand-alone sensor system format, a stand-alone delivery system format, or an integrated sensor and delivery system format. In the case of insulin delivery and glucose sensing, these may be seen as a stand-alone glucose sensor system, a stand-alone insulin delivery system (a so-called insulin pump), or an artificial pancreas. Each such system is adapted to allow for administration of the compound in conjunction with the insulin/other therapeutic active agent administration and/or the sensing of glucose/other analyte by the sensor. The means or device which is included in the system for administration of the compound may the same or different to the device which is included for the administration of the insulin (or other therapeutic active compound).

In the case of an artificial pancreas, this may be designed to administer the compound in conjunction with glucose sensing or insulin administration alone, or in conjunction with both glucose sensing and insulin administration. Various such configurations are depicted in FIGS. 11, 12 and 13. For example where the AP is a wholly SC AP, it may be desirable and advantageous to use the compound in both contexts. However, in another embodiment it may be desirable to administer the compound only in conjunction with the SC insulin administration, or only in conjunction with glucose sensing (see FIGS. 5-10). In the case of a mixed AP, which for example combines IP insulin administration with SC glucose sensing, the compound may be administered only in conjunction with the glucose sensor. However, in other mixed systems, the compound may be administered in conjunction both with glucose sensing and with insulin administration, or only with insulin administration. Still further, a fully IP AP may allow for administration of the compound both in conjunction with glucose sensing and with insulin administration, wherein the compound is administered IP in the vicinity of the site of glucose sensing and in the vicinity of the insulin administration

Accordingly, in one aspect, the artificial pancreas, also referred to herein as an integrated system for controlling the blood glucose level in a subject with diabetes, is configured to administer the compound in conjunction with the sensing of glucose by the glucose sensor. Thus, the system comprises an insulin delivery system and a compound delivery system, as indicated above, which may be the same or different, and a glucose sensor system, which comprises one or more glucose sensors. Based at least in part on the sensor data from the glucose sensor(s), which provides information on the blood glucose level, the control system of the AP determines a dose of insulin to be administered and controls the insulin delivery device to administer the insulin. If desired, further data or information may also be used to determine the insulin dose, for example pre-programmed data or information, or data or information inputted by the user. The control system may be further configured to control the compound delivery device to administer the compound to a site in the vicinity of a glucose sensor in temporal coordination with operation of the glucose sensor to determine the blood glucose level. As noted above, the compound acts to improve blood flow in the vicinity of the glucose sensor. The flow of blood to the vicinity of the sensor may be improved, and this may improve the performance of the sensor. Alternatively, the delivery of the compound may be controlled by means of a slow release system for the compound, which is comprised in, or provided with, the device.

The details discussed and described above in relation to the vicinity, and the temporal coordination, and the dose of the compound etc., are all applicable here, in the context of any system described herein.

If desired, the control system may further be configured to control the compound delivery device to administer the compound at a site in the vicinity of the insulin administration site in temporal coordination with the administration of insulin.

Still further, if desired, the control system may further be configured to determine a therapeutic dose of the compound to administer to the subject to increase the level of blood glucose of the subject based at least on the sensor data, and to control the compound delivery device to administer said therapeutic dose of the compound to the subject to counteract hypoglycaemia or predicted hypoglycaemia.

In various representative embodiments, the integrated system, or artificial pancreas is:
(i) a subcutaneous system wherein the glucose sensors are subcutaneous and the compound and insulin are administered sub-cutaneously; or
(ii) an intraperitoneal system wherein the glucose sensors are intraperitoneal and the compound and insulin are administered intraperitoneally; or
(iii) a combined subcutaneous/intraperitoneal system, wherein the glucose sensors are sub-cutaneous, the compound is administered sub-cutaneously in the vicinity of the glucose sensor site, and insulin is administered intraperitoneally, optionally wherein the compound is administered intraperitoneally in conjunction with the insulin administration.

In any such embodiment the system may further comprise the optional administration of the compound, intraperitoneally or sub-cutaneously, to treat or prevent hypoglycaemia. In the case of (iii), the compound may be administered sub-cutaneously, for example at the site in the vicinity of the glucose sensor, to treat or prevent hypoglycaemia. Thus, the compound delivery system in such a case may be configured and controlled by the control system to perform two separate functions, to administer the compound in conjunction with glucose sensing by the sensor, and separately to administer a therapeutic dose of the compound, when needed or desired to treat or prevent hypoglycaemia.

Analogously, the various elements and system parts discussed above may be presented within a sensor system for determining the level of glucose in the blood of a subject, or in an insulin delivery system for the administration of insulin to a subject.

More broadly, it can be seen, as indicated above that analogous sensor systems can be provided for the detection of any analyte in the blood of a subject, or for the delivery of any therapeutic active agent to a subject.

The doses and administration routes of the compound and, where appropriate, other therapeutic agent may be as discussed above. Thus, for example, the concentration and/or volume and/or rate of administration (e.g. speed) of the compound administered may be determined, and altered or adjusted, to achieve the required effect.

For example, it is known in the art that a CGM may not perform well in the first hours after insertion. Thus, it may be desirable or appropriate to take account of this when setting the parameters of the administration. Furthermore, in analogy to this, the rate, concentration and volume of administration of the compound may influence the performance of the device. It would be a routine matter to determine and take into account the possible negative short-term effect of the administration of the compound on the sensor when it is first used in conjunction with the compound.

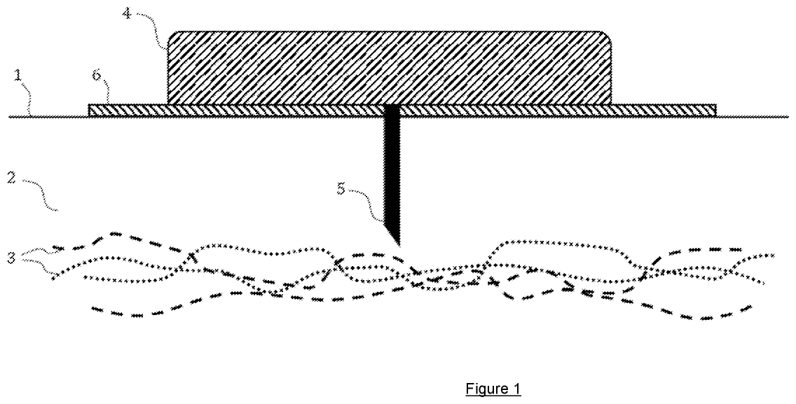

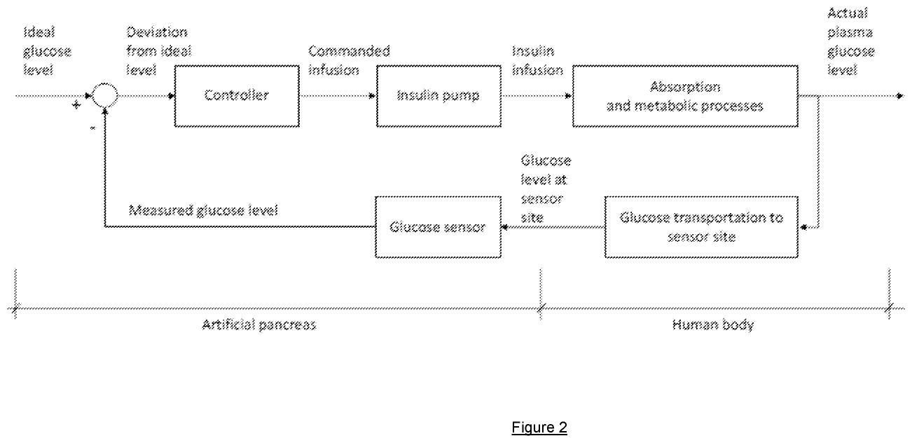

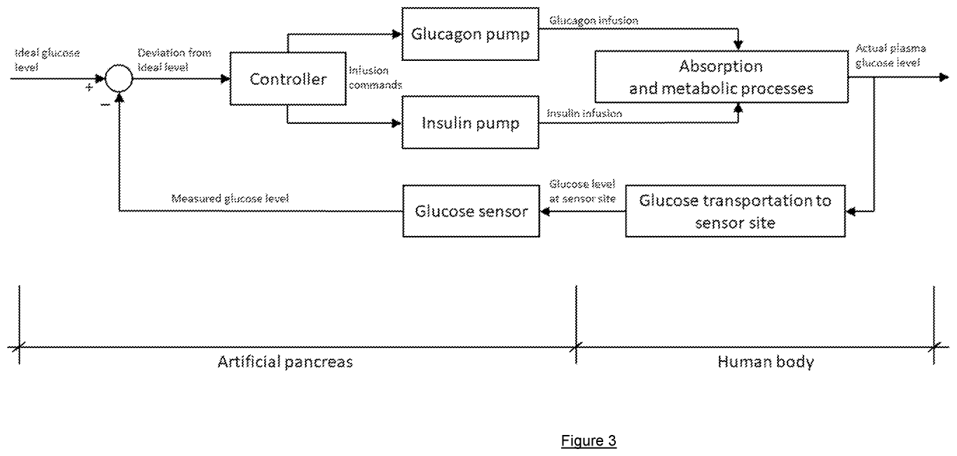

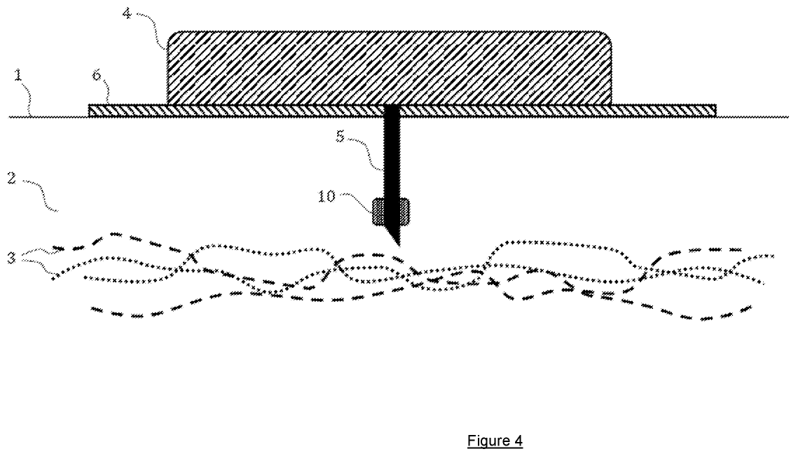

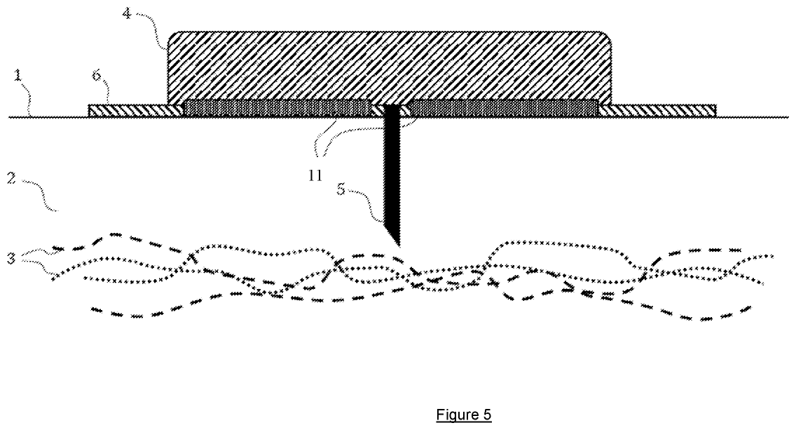

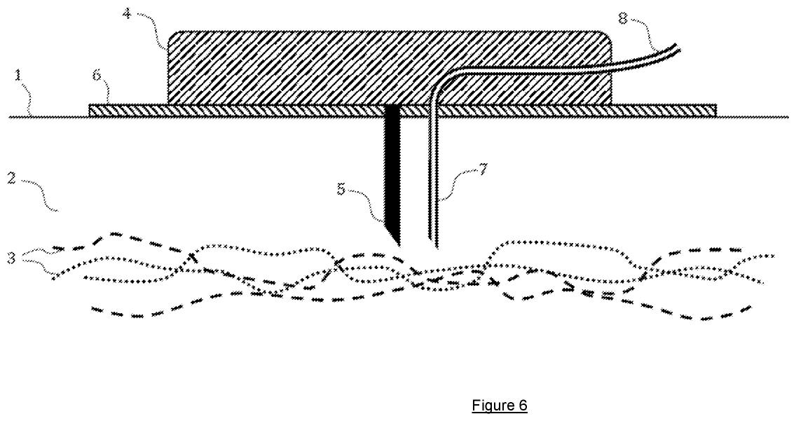

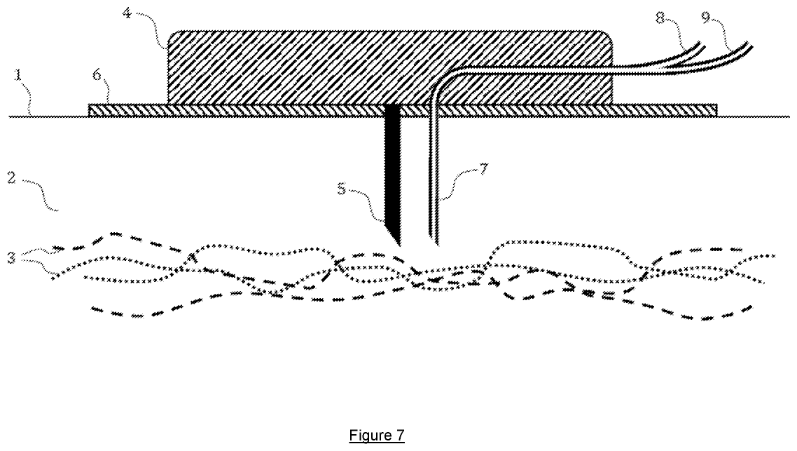

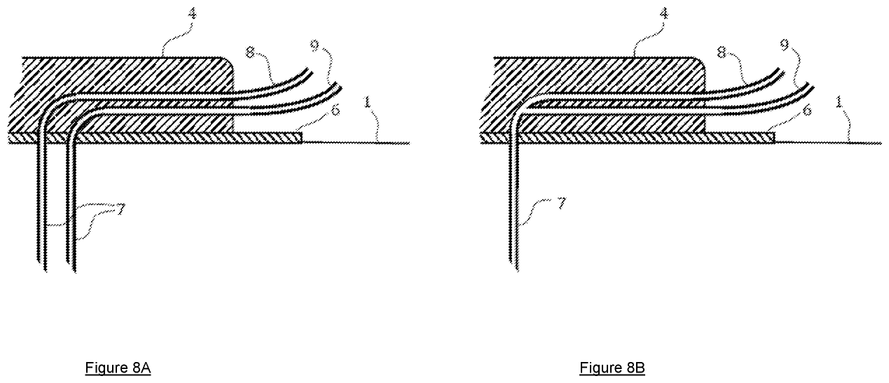

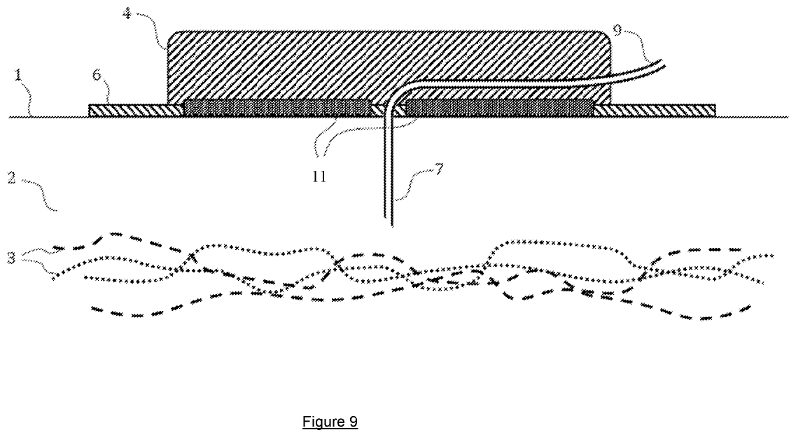

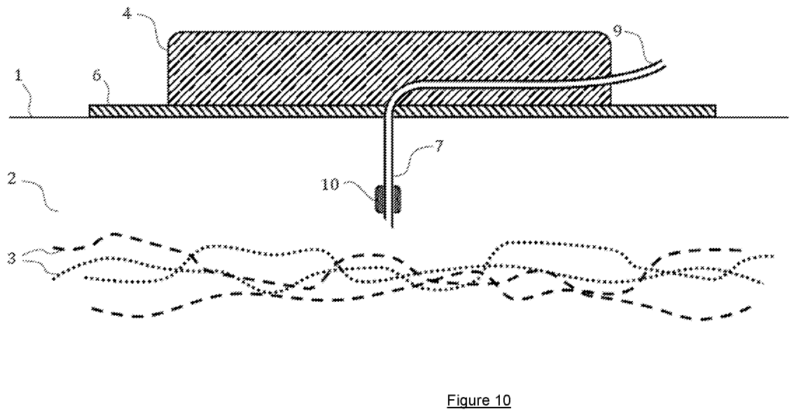

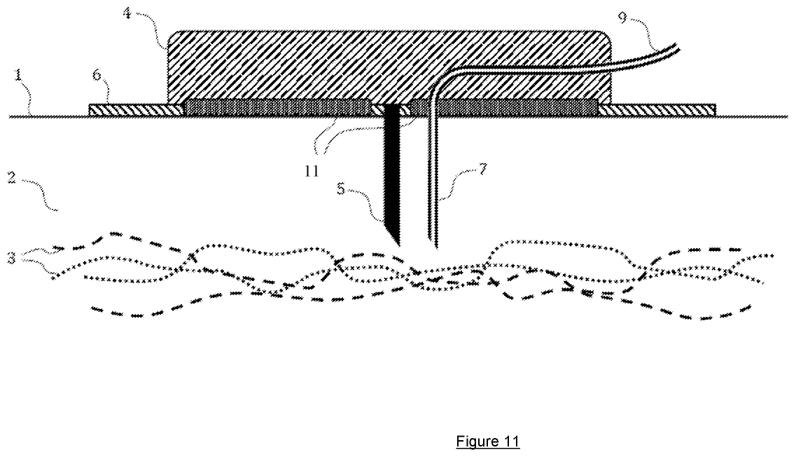

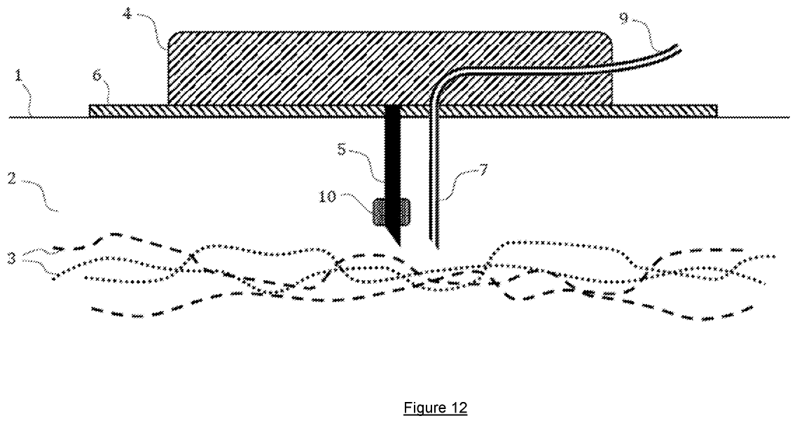

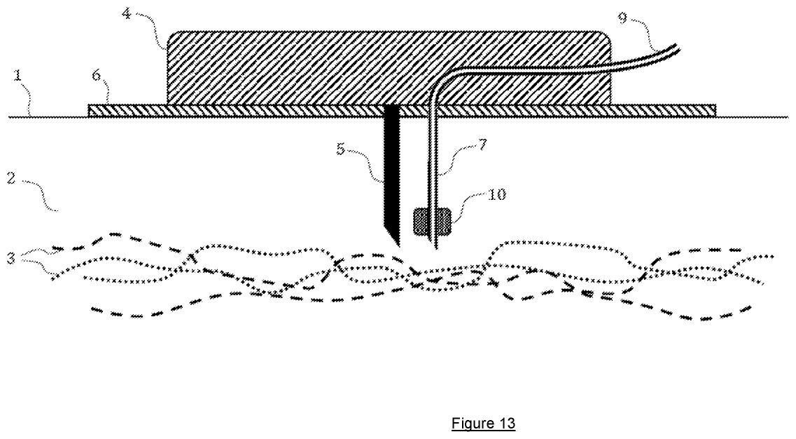

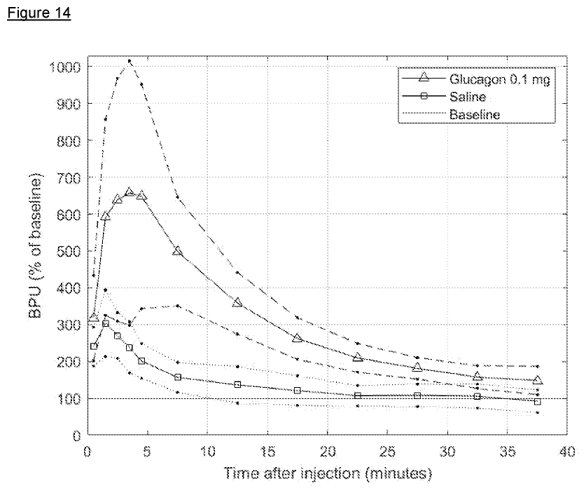

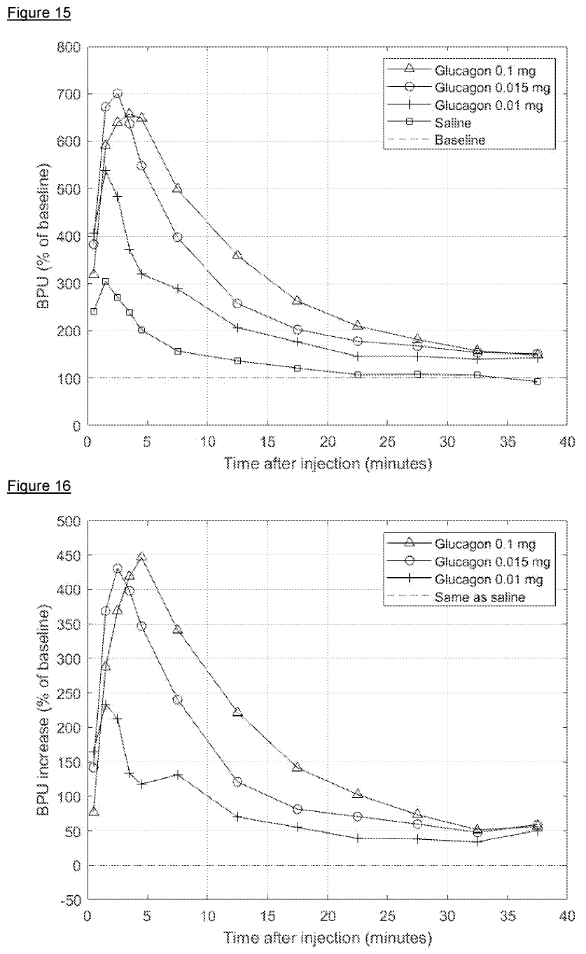

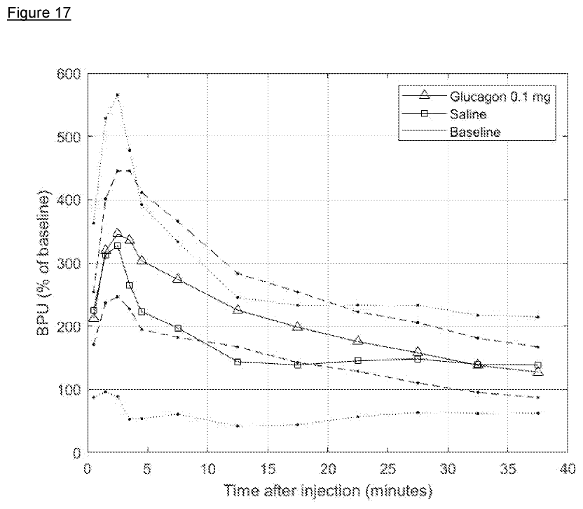

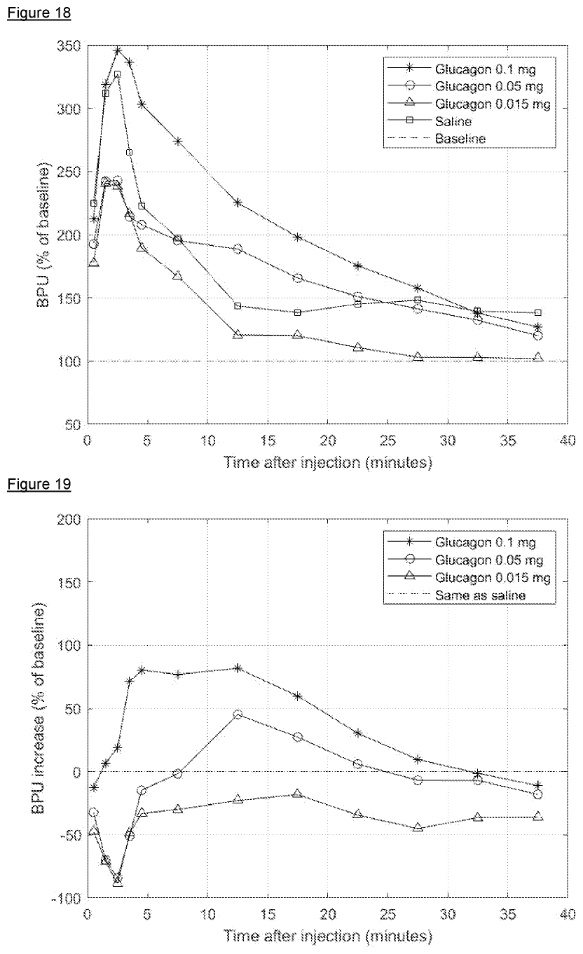

FIG. 1 is a schematic of a typical CGM device, as known in the art. Such a device comprises a housing 4 which is retained against the surface 1 of the skin using an adhesive backing or adhesive pad 6. The device further comprises a glucose sensor 5 which extends through the surface 1 of the skin, into the subcutis 2 and towards the capillaries 3. The glucose sensor 5 is a needle-like electrode which is electrically connected to electronics located within the housing 4. The glucose sensor 5 in this example comprises a platinum-iridium wire (forming a working electrode), with an immobilized mediator and enzyme on the surface. A silver/silver chloride wire wrapped around the working electrode forms a counter electrode. The enzymatic electrode catalyses a reduction-oxidation reaction of glucose, and the resultant movement of electrons produces a current or voltage at the glucose sensor 5 with a magnitude dependent on the concentration of glucose (i.e. the glucose level) in the interstitial fluid. The concentration of glucose in the interstitial fluid can be converted into a corresponding concentration of glucose in blood plasma.

Other forms of glucose sensor comprising a needle-like electrode are of course known and may be used in place of the configuration described above Moreover, other types of glucose sensor which do not include a needle-like electrode are also known and may be used in place of the configuration described above. For example, subcutaneous implants are available which use other technologies (for example, fluorescence, osmotic pressure or other techniques) to measure glucose in the interstitial fluid.

Such standard CGM devices can form part of an artificial pancreas, and provide measurements of a user's glucose levels to a controller of the artificial pancreas. In response to the measured glucose levels, the artificial pancreas regulates the glucose concentration in a user's body by controlling the administration of one or more hormones (or other substances) to the user. Administration of the hormone(s) is generally sub-cutaneous or intra-peritoneal, but can also be intravenous or intraarterial, via lines connected to one or more corresponding hormone pumps comprising a reservoir of the hormone preparation. Artificial pancreases may be mono-hormonal (capable of administering insulin only) or bi-hormonal (capable of administering glucagon or another hormone or another substance, as well as insulin).

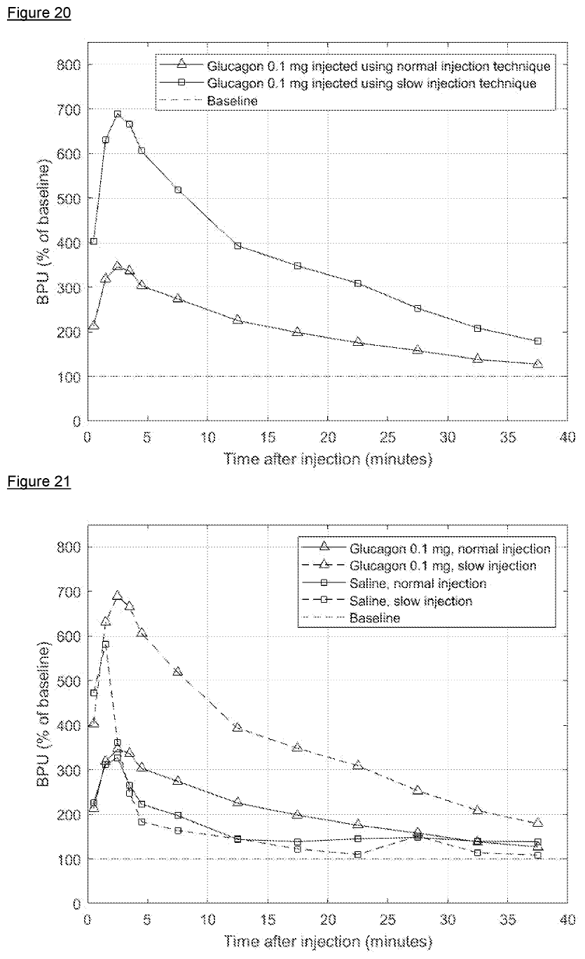

FIG. 2 is a flow chart showing a control loop for a conventional mono-hormonal artificial pancreas. The mono-hormonal artificial pancreas comprises a glucose sensor (such as the CGM device of FIG. 1) at a sub-cutaneous or inter-peritoneal site, a controller, and an insulin pump which administers insulin (under control of the controller) at a site for insulin administration (for example, via an infusion needle at a sub-cutaneous or inter-peritoneal site). Typically, the glucose sensor and the site for insulin administration are spaced apart by at least 4 to 5 cm on the user's body, and may be spaced even further apart.

The CGM device generally measures the glucose level at the sensor site at pre-set time intervals, for example every 5 minutes. Each glucose measurement taken by the CGM device is received by the artificial pancreas controller, which compares the measured glucose level to the ideal glucose level, to determine the deviation (difference) between the two values. On the basis of the determined deviation of the measured glucose level from the ideal glucose level, the controller determines if insulin should be administered. If the controller determines that insulin should be administered, the controller controls the insulin pump to administer insulin via an insulin line and infusion needle located at the site for insulin administration. Absorption of the insulin and metabolic processes utilizing the insulin affect the glucose level in blood plasma. Transport of glucose throughout the body means that a change in the glucose level in blood plasma correspondingly affects the glucose level at the sensor site (in interstitial fluid).

FIG. 3 is a flow chart showing a control loop for a conventional bi-hormonal artificial pancreas using insulin and glucagon. As noted above, conventional bi-hormonal artificial pancreases which administer insulin and a hormone other than glucagon or another substance are also known, but this discussion focuses on a conventional bi-hormonal artificial pancreas which administers insulin and glucagon.

The bi-hormonal artificial pancreas comprises a glucose sensor (such as the CGM device of FIG. 1) at a sub-cutaneous or inter-peritoneal site, a controller, an insulin pump which administers insulin (under control of the controller) at a site for insulin administration (for example, via an infusion needle at a sub-cutaneous or intra-peritoneal site), and a glucagon pump which administers glucagon (under control of the controller) at a site for glucagon administration (for example, via an infusion needle at a sub-cutaneous or intra-peritoneal site). Typically, the glucose sensor, the site for insulin administration and the site for glucagon administration are each spaced apart from each other by at least 4 to 5 cm on the user's body, and may be spaced even further apart.

The CGM device generally measures the glucose level at the sensor site at pre-set time intervals, for example every 5 minutes. Each glucose measurement determined by the CGM device is sent to the artificial pancreas controller, which compares the measured glucose level to the ideal glucose level, to determine the deviation (difference) between the two values. On the basis of the determined deviation of the measured glucose level from the ideal glucose level, the controller determines if insulin or glucagon should be administered. Here, any glucagon administered by a conventional bi-hormonal artificial pancreas is administered for the purpose of reversing an episode of hypoglycaemia (i.e. a therapeutic dose). If the controller determines that insulin or glucagon should be administered, the controller controls either the insulin pump or glucagon pump respectively to administer either insulin (via an insulin line and infusion needle located at the site for insulin administration) or glucagon (via a glucagon line and infusion needle located at the site for glucagon administration).

Absorption of the insulin or glucagon and metabolic processes utilizing the insulin or glucagon affect the glucose level in blood plasma. Transport of glucose throughout the body means that a change in the glucose level in blood plasma correspondingly affects the glucose level at the sensor site (in interstitial fluid).

Conventional bi-hormonal artificial pancreases administer one or the other of insulin and glucagon at any one time; there is no control mechanism by which insulin and glucagon can be administered simultaneously, or in temporal coordination. This is the case because glucagon has opposing effects to insulin; glucagon is administered to raise glucose levels, whereas insulin is administered to lower glucose levels, and hence they are conventionally not administered at or around the same time.

FIG. 4 shows a modified CGM device. The structure of the device is broadly as set out in respect of the device shown in FIG. 1, except that the modified device shown in FIG. 4 further comprises a sub-cutaneous slow-release glucagon “reservoir” 10. The reservoir 10 may take any form which allows for slow-release of glucagon, including for example a slow-release glucagon coating on the glucose sensor 5, or a slow-release glucagon depot implant or micro-ampoule (e.g. a capsule, which encloses or encapsulates a formulation of the glucagon in a manner which permits slow release of the glucagon, for example through the wall of the capsule, or a pill in which glucagon is mixed with one or more other substances to facilitate slow release of the glucagon when placed in the body) located in the vicinity (for example, in close proximity, such as within 2 cm) of the glucose sensor. Slow release carriers and coatings and materials suitable for such use are known in the art, and include for example various polymeric materials.

The subcutaneous slow-release glucagon reservoir 10 is provided in the vicinity of, for example in close proximity (e.g. within 2 cm) to the site of sensing on the glucose sensor 5, near the end of the sensor which is furthest from the skin surface 1. Due to the vasodilatory effects of glucagon, slow-release of glucagon in the vicinity (such as in close proximity, e.g. within 2 cm) of the site where glucose concentration is measured leads, for example a quicker result, or more particularly a reduction in a delay, lag or latency in the sensing, and/or in a more accurate result.

FIG. 5 also shows a CGM device comprising a slow-release glucagon reservoir. In this case, the glucagon reservoir is a transdermal slow-release glucagon patch 11. The transdermal slow-release glucagon patch 11 is adhered to the skin surface 1, within the housing 4 of the CGM device. In general, slow-release transdermal patches are known in the art (and could be modified as necessary in order to deliver glucagon), but to the inventors' knowledge, it is not known to provide a slow-release transdermal glucagon patch as part of a CGM device. Due to the vasodilatory effects of glucagon, slow-release of glucagon in the vicinity (such as in close proximity, e.g. within 2 cm) of the site where glucose concentration is measured leads to faster sensing, for example a quicker result, or more particularly a reduction in a delay, lag or latency in the sensing, and/or in a more accurate result.

Where slow-release reservoirs of glucagon are provided (as for example in FIGS. 4 and 5), the rate of administration of glucagon is such that the dose of glucagon is typically a smaller dose than would be administered in order to treat or to counteract hypoglycaemia (i.e. a therapeutic dose).

The CGM device of FIG. 6 has a structure similar to that shown in FIG. 1 (comprising a housing 4, a glucose sensor 5, and an adhesive patch 6). However, the device shown in FIG. 6 further comprises a subcutaneous glucagon injection device comprising an infusion needle 7 in fluidic communication with a glucagon infusion line 8. The infusion needle 7 is located in in the vicinity of, or in close proximity to the glucose sensor 5 (e.g. within 2 cm). The glucagon infusion line 8 is in fluidic communication with a glucagon pump (not shown) comprising a glucagon reservoir. The glucagon pump is located outside of the body. Provision of a glucagon pump (instead of a slow-release glucagon reservoir) allows for glucagon to be delivered in a controlled dose at a predetermined time. The administration of glucagon by the glucagon pump is under the control of a controller (not shown). The time at which the glucagon is administered is in temporal coordination with the sensing of glucose by the sensor. In particular, the glucagon is administered simultaneously with the time of sampling by the sensor, or at least within 30 minutes before sampling occurs. Due to the vasodilatory effects of glucagon, administration of glucagon in the vicinity (such as in close proximity, e.g. within 2 cm) of the site where glucose concentration is measured leads to faster sensing, for example a quicker result, or more particularly a reduction in a delay, lag or latency in the sensing, and/or in a more accurate result. Each dose of glucagon is typically a smaller dose than would be administered in order to treat or to counteract hypoglycaemia.

FIG. 7 shows a schematic view of an artificial pancreas. The artificial pancreas of FIG. 7 comprises a housing 4, a glucose sensor 5, an adhesive patch 6, and an infusion needle 7. The artificial pancreas further comprises a glucagon infusion line 8 connected to a glucagon pump (not shown) and an insulin infusion line 9 in fluidic communication with an insulin pump (not shown). The insulin pump comprises an insulin reservoir and is located outside of the body. The glucagon pump comprises a glucagon reservoir and is located outside of the body. In this embodiment, the insulin infusion line 9 converges to the glucagon infusion line 8, so that both insulin and glucagon can be delivered into the body via a single infusion needle 7. The infusion needle 7 is located in the vicinity of, or in close proximity to, the glucose sensor 5 (e.g. within 2 cm). Administration of the glucagon and insulin is under the control of a controller (not shown).

Provision of a glucagon pump allows for glucagon to be delivered in a controlled amount at a predetermined time. The time at which the glucagon is administered is in temporal coordination with the sensing of glucose by the sensor and/or and in temporal coordination with the administration of insulin. In particular, the glucagon is administered shortly before the time of sampling by the sensor, or at least within 30 minutes before sampling occurs. Moreover, the glucagon is administered simultaneously with the time of administering insulin, or at least within 30 minutes before or 1-2 hours after administering insulin. Each dose of glucagon is typically a smaller dose than would be administered in order to treat or to counteract hypoglycaemia.

As discussed above, due to the vasodilatory effects of glucagon, administration in the vicinity (such as in close proximity, e.g. within 2 cm) of the site where glucose concentration is measured leads to faster sensing, for example a quicker result, or more particularly a reduction in a delay, lag or latency in the sensing, and/or in a more accurate result. Moreover, due to the vasodilatory effects of glucagon, administration in the vicinity (such as in close proximity, e.g. within 2 cm) of the site where insulin is administered leads to an improved effect from the insulin administration. An improved effect of insulin may accordingly be a faster, or more rapid, decrease of blood glucose level, or a faster onset of a reduction in blood glucose level. It may also lead to a more predictable absorption of insulin and thereby also a more predictable effect on glucose levels by reducing the day to day variation in insulin absorption observed with SC insulin injections.

In addition to administration of glucagon for the purposes of the advantageous vasodilatory effects (in temporal coordination with the administration of insulin and/or sensing of glucose), the glucagon pump may be controlled by the controller to administer a rescue dose of glucagon (a therapeutic dose), in the event that the controller determines that the user is suffering from hypoglycaemia or is at risk of hypoglycaemia in the immediate future.

As noted above, in conventional bi-hormonal artificial pancreases, the sites at which glucose is sensed, insulin is administered and glucagon is administered are spatially separated from each other on the user's body. To the inventors' knowledge, it is not known to provide all three sites in the immediate vicinity together, as shown in FIG. 7.

FIGS. 8A and 8B each show a subcutaneous glucagon and insulin injection device. Each comprises a housing 4 which is retained against the surface 1 of the skin using an adhesive backing or adhesive pad 6. Each further comprises a glucagon infusion line 8 and an insulin infusion line 9. In FIG. 8A, two infusion needles 7 are provided—a first infusion needle is in fluidic communication with the glucagon infusion line 8, and a second infusion needle is in fluidic communication with the insulin infusion line 9. The two infusion needles are located in the vicinity of one another, for example in close proximity to each other (e.g. within 2 cm). In comparison, only one infusion needle 7 is provided in the device shown in FIG. 8B, and this single infusion needle is in fluidic communication with both the glucagon infusion line 8 and insulin infusion line 9. In both FIGS. 8A and 8B, the glucagon infusion line 8 is in fluidic communication with a glucagon pump (not shown) comprising a glucagon reservoir and the insulin infusion line 9 is in fluidic communication with an insulin pump (not shown) comprising an insulin reservoir. The insulin pump and glucagon pump are located outside of the body.

The subcutaneous glucagon and insulin injection devices shown in FIGS. 8A and 8B comprise, or are in communication with, a controller which controls administration of glucagon and insulin in response to measurements of blood glucose levels from a separate CGM device. Glucagon may be administered in order to affect the glucose level in the body (i.e. glucagon may be administered in order to treat or to counteract hypoglycaemia), and/or to enhance the absorption of subcutaneous insulin from the insulin infusion line 9 delivered by the infusion needle 7.

Provision of a glucagon pump allows for glucagon to be delivered in a controlled dose at a predetermined time. The time at which the glucagon is administered is in temporal coordination with the administration of insulin. In particular, the glucagon is administered simultaneously with the time of administering insulin, or at least within 30 minutes before or within 2 hours after administering insulin.

Due to the vasodilatory effects of glucagon, administration in the vicinity (such as in close proximity, e.g. within 2 cm) of the site where insulin is administered leads to an improved effect from the insulin administration. An improved effect of insulin may accordingly be a faster, or more rapid, decrease of blood glucose level, or a faster onset of a reduction in blood glucose level. It may also lead to a more predictable absorption of insulin and thereby also a more predictable effect on glucose levels by reducing the day to day variation in insulin absorption observed with SC insulin injections. Each dose of glucagon is typically a smaller dose than would be administered in order to treat or to counteract hypoglycaemia.

Conventionally, the sites at which insulin is administered and glucagon is administered are spatially separated on the user's body. To the inventors' knowledge, it is not known to provide both sites in the immediate vicinity together, as shown in FIGS. 8A and 8B.

FIG. 9 shows schematically an insulin injection device comprising a housing 4 which is retained against the surface 1 of the skin using an adhesive backing or adhesive pad 6. The device further comprises an infusion needle 7 in fluidic communication with an insulin infusion line 9, which is connected to an insulin pump (not shown) comprising an insulin reservoir. The insulin pump is located outside of the body. The insulin injection device of FIG. 9 further comprises a transdermal slow-release glucagon patch 11. The transdermal slow-release glucagon patch 11 is adhered to the skin surface 1, within the housing 4 of the insulin injection device. In general, slow-release transdermal patches are known in the art, but to the inventors' knowledge, it is not known to provide a slow-release transdermal glucagon patch as part of an insulin injection device.

FIG. 10 shows an insulin injection device similar to that shown in FIG. 9, except that in the device of FIG. 10, a subcutaneous slow-release glucagon reservoir 10 is provided, instead of a transdermal slow-release glucagon patch 11. The slow-release glucagon reservoir 10 is provided in close proximity to the end of the infusion needle 7, in the vicinity of the site of administration of the insulin (such as in close proximity, for example within 2 cm). The reservoir 10 may take any form which allows for slow-release of glucagon, including for example a slow-release glucagon coating on the infusion needle 7, or a slow-release glucagon depot implant or micro-ampoule located in the vicinity (such as in close proximity, for example, within 2 cm) of the end of the infusion needle 7. Slow release carriers and coatings and materials suitable for such use are known in the art, and include for example various polymeric materials.

In respect of both insulin injection devices shown in FIGS. 9 and 10, the insulin injection device comprises, or is in communication with, a controller which controls administration of insulin in response to measurements of blood glucose levels from a CGM device. Due to the vasodilatory effects of glucagon, administration of glucagon in the vicinity (such as in close proximity, e.g. within 2 cm) of the site where insulin is administered leads to an improved effect from the insulin administration. An improved effect of insulin may be a faster, or more rapid, decrease of blood glucose level, or a faster onset of a reduction in blood glucose level. It may also lead to a more predictable absorption of insulin and thereby also a more predictable effect on glucose levels by reducing the day to day variation in insulin absorption observed with SC insulin injections.

FIGS. 11, 12 and 13 each show schematically an artificial pancreas. In each case, the artificial pancreas comprises a housing 4 which is retained against the surface 1 of the skin using an adhesive backing or adhesive pad 6. The artificial pancreases shown in FIGS. 11, 12 and 13 each further comprise a glucose sensor 5 which extends through the surface 1 of the skin, into the subcutis 2 and towards capillaries 3. As described above (with reference to the device shown in FIG. 1), the glucose sensor 5 is a needle-like electrode which is electrically connected to electronics located within the housing 4.

The artificial pancreases shown in FIGS. 11, 12 and 13 each further comprise an infusion needle 7 in fluidic communication with an insulin infusion line 9, which is connected to an insulin pump (not shown) comprising an insulin reservoir. The insulin pump is located outside of the body. As described in more detail below, each of the artificial pancreases shown in FIGS. 11, 12 and 13 further comprises a means for slow-release of glucagon in in the vicinity (such as in close proximity, e.g. within 2 cm) of the site where glucose concentration is measured by the glucose sensor 5, and where insulin is administered by the infusion needle 7.

In the device shown in FIG. 11, the means for slow release of glucagon is a transdermal slow-release glucagon patch 11 (as discussed above in relation to FIGS. 5 and 9). The transdermal slow-release glucagon patch 11 is adhered to the skin surface 1, within the housing 4 of the artificial pancreas.

In the devices shown in FIGS. 12 and 13, the means for slow release of glucagon is a subcutaneous slow-release glucagon reservoir 10. In the device shown in FIG. 12, the subcutaneous slow-release glucagon reservoir 10 is provided in the vicinity (such as in close proximity to, e.g. within 2 cm) of the end of the glucose sensor 5 which is furthest from the skin surface 1, in the vicinity of the point of actual glucose sensing on the glucose sensor 5. In the device shown in FIG. 13, the subcutaneous slow-release glucagon reservoir 10 is provided in the vicinity (such as in close proximity to, e.g. within 2 cm) of the end of the infusion needle 7, close to the site of administration of the insulin.

The reservoirs 10 of FIGS. 12 and 13 may take any form which allows for slow-release of glucagon, including for example a slow-release glucagon coating, or a slow-release glucagon depot implant or micro-ampoule. Slow release carriers and coatings and materials suitable for such use are known in the art, and include for example various polymeric materials.

In respect of all of the artificial pancreases shown in FIGS. 11, 12 and 13, the artificial pancreas comprises a controller which controls the administration of insulin in response to measurements of blood glucose levels from the glucose sensor. The set ups in FIGS. 11, 12 and 13 may also be used when glucose sensing and insulin infusion by an insulin pump is used as stand-alone solutions and not as integrated parts of an artificial pancreas.

Due to the vasodilatory effects of glucagon, administration of glucagon in the vicinity (such as in close proximity, e.g. within 2 cm) of the site where insulin is administered leads to an improved effect from the insulin administration. An improved effect of insulin may be a faster, or more rapid, decrease of blood glucose level, or a faster onset of a reduction in blood glucose level. It may also lead to a more predictable absorption of insulin and thereby also a more predictable effect on glucose levels by reducing the day to day variation in insulin absorption observed with SC insulin injections. Moreover, administration in the vicinity (such as in close proximity, e.g. within 2 cm) of the site where glucose concentration is measured leads to faster sensing, for example a quicker result, or more particularly a reduction in a delay, lag or latency in the sensing, and/or in a more accurate result.

All of the devices shown in FIGS. 3 to 13 enable administration of glucagon in in the vicinity of or close proximity to the location where glucose levels are sensed and/or in the vicinity of or close proximity to the location where insulin is administered (and absorbed). In the vicinity may mean with 3 cm, or within 2.5 cm, for example. Close proximity may be within 2 cm, for example within 1.5 cm, or within 1 cm. The administration of glucagon is highly localised, such that whilst the dose of glucagon is typically a smaller dose than would be administered as a therapeutic dose in order to treat or to counteract hypoglycaemia, in the localised region around the site of administration of glucagon, the concentration of glucagon may be higher that the corresponding concentration resulting from administration of a therapeutic dose of glucagon. When the traditional 1 mg therapeutic dose to counteract or treat hypoglycaemia has been absorbed to the blood it is diluted by approximately 5 litres of blood in an average person. In comparison, the dose administered in the foregoing devices for the purposes of vasodilation may initially be distributed in a subcutaneous volume of approximately or less than 20 cm³. The subcutaneous tissue comprises around 90% cells and 10% interstitial fluid, so in a 20 cm³ subcutaneous volume the glucagon is diluted by only approximately 2 cm³ fluid.

The invention will now be described in further detail in the Examples below.

### Example 1

### Effect of Glucagon on Blood Flow

An experiment was performed on 2 young healthy adult human volunteers. The effect of subcutaneous (hereinafter, SC) injections on blood flow in the skin were tested 6 times in the 2 subjects (4 tests in a male subject and 2 tests in a female subject). The glucagon was administered by SC injection of 0.1 ml of 1 mg/ml glucagon (i.e. an injection containing 0.1 mg of glucagon) to sites on the lateral side of both upper arms of the subjects. Blood flow was measured on the skin surface at the site of administration by laser Doppler technology. As a control the same volume of physiological saline (0.9%) was injected as a placebo at the same sites.

The results of this experiment are presented in FIG. 14 for both glucagon and placebo. The average of the 6 tests is shown, together with 95% confidence intervals. It can be seen that the effect of glucagon on local blood flow was massive, with the flow increasing several hundred percent. Even after returning to “normal”, the blood flow is increased compared to before the glucagon injection. Injections with the same volume of physiological saline had only a short-lived acute and no long-term effect.

This effect was further investigated in a similar experiment with varying doses of glucagon. Again, the same 2 subjects were used, and SC injections of glucagon or placebo were given 6 times (4 tests in a male subject and 2 tests in a female subject). The glucagon was administered to sites on the lateral sides of both upper arms in amounts of 0.1 mg, 0.015 mg and 0.01 mg (0.1 ml of glucagon at concentrations of 0.1 mg/ml, 0.015 mg/ml and 0.01 mg/ml, respectively). Blood flow was measured on the skin surface at the site of administration by laser Doppler technology. As a control the same volume of physiological saline (0.9%) was injected as a placebo at the same sites.

The results of this experiment are presented in FIG. 15, which shows the average of the 6 tests for each concentration of glucagon and for placebo. Equivalent results are also shown in FIG. 16 with the placebo effect subtracted. It can be seen that a dramatic increase in local blood flow (in the order of 400-450% of the baseline reading) was obtained with both 0.1 mg and 0.015 mg glucagon. A similar, although less pronounced, effect was also observed with 0.01 mg glucagon. As in the first experiment, the increase in blood flow was found to persist even once the value had stabilised. This was seen with all doses of glucagon, but not with the placebo.

Further experiments were conducted to investigate the effect on local blood flow of subcutaneous injections of glucagon in the abdomen. Again, 2 subjects were used, and SC injections of glucagon or placebo were given 6 times (3 tests in a male subject and 3 tests in a female subject). The glucagon was administered by SC injection of 0.1 ml of 1 mg/ml glucagon (i.e. an injection containing 0.1 mg of glucagon) to sites on both sides of the abdomen of the subjects. Blood flow was measured on the skin surface at the site of administration by laser Doppler technology. As a control the same volume of physiological saline (0.9%) was injected as a placebo at the same sites.

The results of this experiment are presented in FIG. 17 for both glucagon and placebo. The average of the 6 tests is shown, together with 95% confidence intervals. It can be seen that the administration of 0.1 mg glucagon resulted in a significant increase in local blood flow, which was slightly greater than that observed when saline was administered. The increase in local blood flow following administration of glucagon was also more persistent than that observed following administration of the control, though the blood flow values for both groups were approximately equal after around 30 minutes.

This effect was further investigated in a similar experiment with varying doses of glucagon. Again, 2 subjects were used, and SC injections of glucagon or placebo were given 6 times (3 tests in a male subject and 3 tests in a female subject). The glucagon was administered to sites on both sides of the abdomen in amounts of 0.1 mg, 0.05 mg and 0.015 mg (0.1 ml of glucagon at concentrations of 0.1 mg/ml, 0.05 mg/ml and 0.015 mg/ml, respectively). Blood flow was measured on the skin surface at the site of administration by laser Doppler technology. As a control the same volume of physiological saline (0.9%) was injected as a placebo at the same sites.

The results of this experiment are presented in FIG. 18, which shows the average of the 6 tests for each concentration of glucagon and for placebo. Equivalent results are also shown in FIG. 19 with the placebo effect subtracted. When the glucagon was administered to the abdomen, only 0.1 mg glucagon produced an initial increase in local blood flow greater than that seen following the administration of the same control. The administration of 0.05 mg glucagon did not result in a significant increase in local blood flow initially, but the increase that was observed was more persistent than the effect of the saline injection, such that after 15 minutes the local blood flow was higher than that observed at the same time after the control injection. The administration of 0.015 mg glucagon to the abdomen did not appear to have a significant effect on local blood flow, relative to the control injection.

Further experiments were conducted to investigate the effect on local blood flow of subcutaneous injections of glucagon in the thigh. A single female subject was used, and SC injections of glucagon or placebo were given 7 times. The glucagon was administered by SC injection of 0.05 mg of glucagon (5 times with 0.1 ml and 2 times with 0.05 ml) to sites on the thighs of the subject. Blood flow was measured on the skin surface at the site of administration by laser Doppler technology. As a control the 0.9% saline (5 times with 0.6 ml and 2 times with 0.03 ml) was injected as a placebo at the same sites.

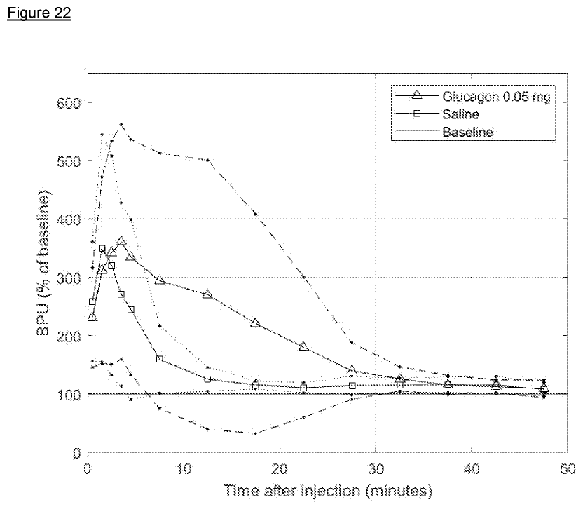

The results of this experiment are presented in FIG. 22 for both glucagon and placebo. The average of the 7 tests is shown, together with 95% confidence intervals. It can be seen that the administration of 0.05 mg glucagon resulted in a significant increase in local blood flow, which was slightly greater than that observed when saline was administered. The increase in local blood flow following administration of glucagon was also more persistent than that observed following administration of the control, though the blood flow values for both groups were approximately equal after around 30 minutes.

This effect was further investigated in a similar experiment with varying doses of glucagon. Again, a single female subject was used, and SC injections of glucagon or placebo were given 7 times. The glucagon was administered to sites on the thighs of the subject in amounts of 0.05 mg (5 times in 0.1 ml, 2 times in 0.05 ml), 0.03 mg (5 times in 0.06 ml, 2 times in 0.03 ml) and 0.01 mg (5 times in 0.02 ml, 2 times in 0.01 ml). Blood flow was measured on the skin surface at the site of administration by laser Doppler technology. As a control, 0.9% saline was injected (5 times in 0.06 ml, 2 times in 0.03 ml) as a placebo at the same sites.

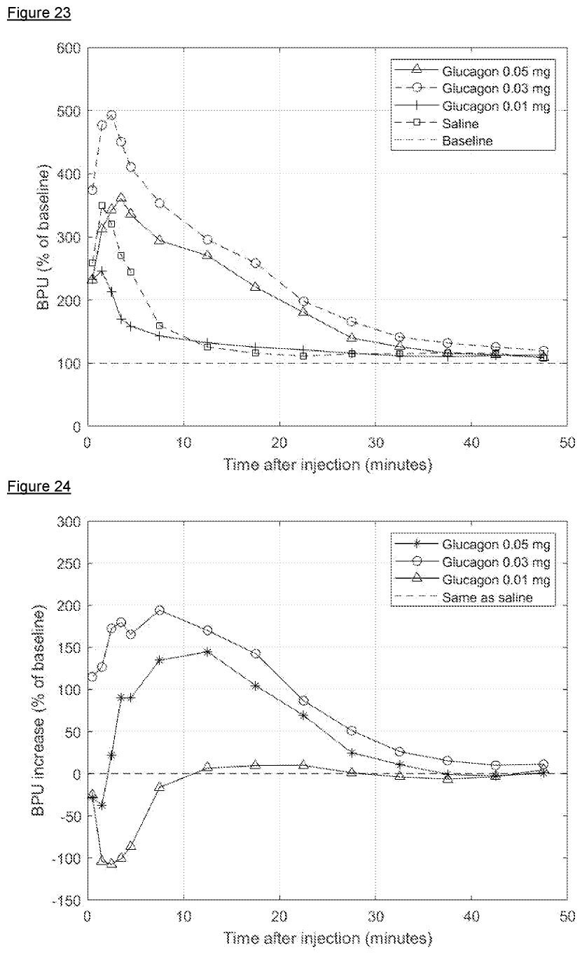

The results of this experiment are presented in FIG. 23, which shows the average of the 7 tests for each concentration of glucagon and for placebo. Equivalent results are also shown in FIG. 24 with the placebo effect subtracted. When the glucagon was administered to the thighs, 0.03 mg glucagon produced the biggest increase in local blood flow. The administration of 0.05 mg glucagon also resulted in a significant increase in local blood flow which was slightly larger than that observed following administration of the saline control, and significantly more persistent. The administration of 0.01 mg glucagon to the thighs did not appear to have a significant effect on local blood flow, relative to the control injection.

This effect was further investigated in a similar experiment to investigate the effect of glucagon on the performance of CGMs. Again, 2 subjects were used showing data from 12 meals in a non-diabetic female and 11 meals in a non-diabetic male collected with Dexcom G6 CGMs. The subjects wore two CGMs placed symmetrically on the lateral side of each overarm. 1-3 minutes before the start of ingesting the meal 0.1 ml of glucagon (1 mg/ml) was injected at the site of one CGM and 0.1 ml of placebo (saline (0.9%) on the contralateral site of CGM. Before each meal, the site of glucagon delivery was decided by a new randomization.

The results of this experiment are presented in FIG. 26. This shows that administration of the glucagon at the site of CGM results in a faster and larger increase in glucose levels detected compared to placebo. This effect can be clearly seen at 10 minutes in one of the subjects.

### Example 2

### Impact of Injection Technique on Glucagon Effect

To observe the impact of injection technique, and particularly speed of injection, on the effect induced by subcutaneous injection of glucagon, an experiment was conducted to compare 2 different injection techniques; one where the injection lasted 1-3 seconds (technique 1), and one where the injection lasted at least 10 seconds (technique 2). 6 injections were given with technique 1 and 4 injections were given with technique 2. In each case, 0.1 mg of glucagon (in a 0.1 ml injection) was injected subcutaneously into the abdomen of the subjects. For each technique, half of the injections were given to a male subject and half to a female subject. Blood flow was measured on the skin surface at the site of administration by laser Doppler technology.

The results of this experiment are presented in FIG. 20. In addition, in FIG. 21 the same results are shown together with the results of placebo injections of 0.1 ml of 0.9% saline using the same 2 techniques. For technique 1, 6 placebo injections were given, and for technique 2, 2 placebo injections were given. Again, half of the injections with each technique were done in a male subject, and half were done in a female subject.

It can be seen that technique 2, where the injection takes place more slowly, resulted in a significantly larger increase in local blood flow. This effect was observed with both glucagon and placebo injections, though notably the effect of the “slow” glucagon injection on local blood flow was more persistent than the effects of any of the other injections.

### Example 3

### Impact of Probe Placement on Measured Glucagon Effect

The impact of the placement of the laser Doppler probe on the measured effect of glucagon injections on local blood flow was investigated by giving injections at multiple sites with varying distances from the probe. The experiment involved the subcutaneous injection of 0.015 mg of glucagon (in 0.1 ml) or 0.1 ml of 0.9% saline as a control at sites on the lateral sides of the upper arms of the subjects. The injections were given either under the probe, 1.6 cm from the probe centre, 3 cm from the probe centre, or 5 cm from the probe centre. In each case, 6 injections were given, except for 1.6 cm from the probe centre where only 4 saline injections were given. In all cases, half of the injections were given to a male subject and half to a female subject, except for under the probe, where 4 tests were done on the male subject and 2 on the female subject. Blood flow was measured on the skin surface by laser Doppler technology.

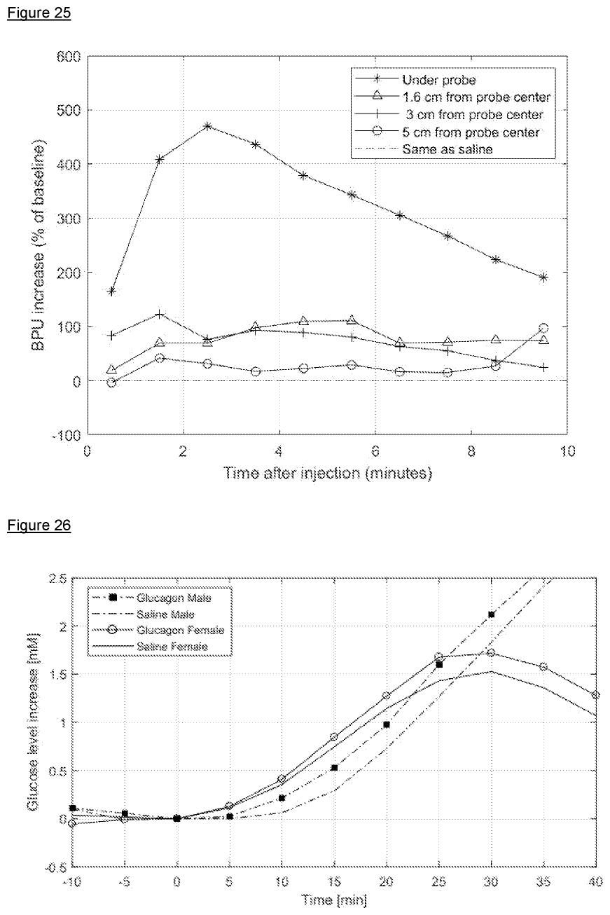

The results of this experiment are presented in FIG. 25, which shows the average of the 6 glucagon injections with the average of the placebo injections subtracted. It can be seen that the largest increase in local blood flow was observed when the injection was given directly under the probe. When the injections were given at greater distances from the probes, the increase in local blood flow was less significant. It did not appear that there was a significant difference between the results obtained when the injection was given at 1.6 cm, 3 cm or 5 cm from the probe centre. However, except for injection 5 cm from the probe the effect was larger than placebo.

1. A compound with glucagon activity for use in the delivery of an active therapeutic agent to a subject and/or in the determination of the blood level of an analyte in the subject, wherein the compound is administered with the active agent and/or in conjunction with determination of the analyte by a bodily sensor, and wherein the compound is administered to the subject at a site which is in the vicinity of the site of administration of the active agent and/or of the site(s) of sensing of the analyte by the bodily sensor, and in temporal coordination with the administration of the active agent and/or with the sensing of the analyte by the sensor.

2. The compound for use according to claim 1, wherein the use is in the treatment and/or management of a subject with diabetes by co-administration with insulin and/or in conjunction with glucose sensing by a glucose sensor.

3. A compound with glucagon activity for use in the delivery of a second active therapeutic agent to a subject, wherein said use comprises co-administering the compound with said second agent, and said compound is administered to the subject in temporal coordination with the second agent at a site which is in the vicinity of the administration site of the second agent.

4. A compound for use according to claim 3, wherein the second active therapeutic agent is insulin, and said compound and insulin are co-administered for use in the treatment of diabetes.

5. A compound with glucagon activity for use in conjunction with a bodily analyte sensor in the determination of the blood level of an analyte in the subject, said use comprising administering said compound at a site in the vicinity of the analyte sensor in temporal coordination with the time of analyte sensing by the sensor, preferably wherein the analyte is glucose and the bodily analyte sensor is a bodily glucose sensor.

6. The compound for use according to any one of claims 1 to 5, wherein the compound is administered at a micro-dose which is less than the dose required to achieve a clinically significant increase in the level of glucose in the blood.

7. The compound for use according to any one of claims 1 to 6, wherein the compound is administered at a dose of no more than 0.1 mg.

8. The compound for use according to any one of claims 1 to 7, wherein the compound is administered at a dose of no more than 0.05 mg.

9. The compound for use according to any one of claims 2, or 4 to 8, wherein the treatment and/or management of the subject further comprises the separate administration of a therapeutic dose of a compound with glucagon activity to increase glucose levels in the subject.

10. The compound for use according to any one of claims 1 to 9, wherein in said use the compound is administered to a site within 3 cm of the site of administration of the second active therapeutic agent and/or of the site(s) of analyte sensing.

11. The compound for use according to any one of claims 1 to 4 or 6 to 10, wherein in said use the compound and the insulin or other second active therapeutic agent are administered separately.

12. The compound for use according to any one of claims 1 to 4 or 6 to 10 wherein in said use the compound and the insulin or other second active therapeutic agent are administered as an admixture.

13. The compound for use according to any one of claims 1 to 12, wherein in said use the compound and the second active therapeutic agent are co-administered in conjunction with or in response to sensing of the analyte by said bodily analyte sensor or by another analyte sensor.

14. The compound for use according to any one of claims 1 to 13, wherein the compound and the insulin or other second active therapeutic agent are each administered subcutaneously or intraperitoneally.

15. The compound for use according to any one of claims 2, or 4 to 14, wherein the compound and/or the insulin are each administered:
(i) from an artificial pancreas, being an integrated device comprising one or more delivery devices or means for administration of the insulin and the compound, and a glucose sensor; or
(ii) from an insulin pump and/or a pump for delivery of the compound, which may be the insulin pump or a different pump; or
(iii) as part of a regime of multiple daily injections; or wherein:
(iv) the compound is administered by continuous infusion in the vicinity of the site of insulin administration or the glucose sensor; or
(v) the compound is administered in the form of a slow release preparation.

16. The compound for use according to claim 15, wherein in part (v) the slow release preparation is a composition or reservoir for administration at a site in the vicinity of the site of insulin administration and/or the vicinity of the glucose sensor; or is in the form of coating at or towards the delivery end of an insulin delivery line; or is a cutaneous adhesive patch for application in the vicinity of the site of insulin administration and/or the vicinity of the glucose sensor.

17. The compound for use according to any one of claims 2, 4, or 6 to 16, wherein the compound is administered subcutaneously and the insulin is administered subcutaneously, intraperitoneally or in any other body cavity or organ.

18. The compound for use according to any one of claims 1, 2, or 5 to 17, wherein the sensors are subcutaneous, intraperitoneal or placed in any other body cavity or organ.

19. The compound for use according to claim 18, wherein the compound is administered subcutaneously in conjunction with subcutaneous glucose sensor(s), and wherein optionally the compound is further separately administered subcutaneously to increase the level of blood glucose when required, optionally at the same site.

20. The compound for use according to claim 19, wherein said use further comprises administering insulin, optionally intraperitoneally or in any other body cavity or organ.

21. The compound for use according to any one of claims 2, or 4 to 20, wherein said compound and said insulin are administered via, and said glucose sensor(s) forms part of, an artificial pancreas, being an integrated system for controlled delivery of said insulin and compound and blood glucose sensing.

22. An integrated system for controlling the blood glucose level in a subject with diabetes comprising:
(i) one or more glucose sensors configured to determine the blood glucose level of the subject and to provide sensor data associated with the blood glucose level;
(ii) a compound delivery means configured to administer a compound with glucagon activity to said subject;
(iii) an insulin delivery device configured to administer insulin to said subject; and
(iv) a control system configured to receive sensor data from the glucose sensor(s) and to determine a dose of insulin to administer to the subject based at least on the sensor data and to control the insulin delivery device to administer said dose, wherein:
(a) the compound delivery means comprises a slow-release reservoir of the compound with glucagon activity, which is configured to administer the compound to a site in the vicinity of the insulin administration site and/or in the vicinity of the glucose sensor; or
(b) the compound delivery means is controllable to administer the compound with glucagon activity to said subject, and the control system is configured to control the compound delivery means to administer the compound to a site in the vicinity of the insulin administration site in temporal coordination with the administration of the insulin, thereby to improve blood flow in the vicinity of the insulin administration site, and/or to control the compound delivery means to administer the compound to a site in the vicinity of a glucose sensor in temporal coordination with operation of the glucose sensor to determine the blood glucose level, thereby to improve blood flow in the vicinity of the glucose sensor.

23. The integrated system of claim 22, wherein the control system is further configured to determine a therapeutic dose of the compound to administer to the subject to increase the level of blood glucose of the subject based at least on the sensor data, and to control the compound delivery means to administer said therapeutic dose of the compound to the subject to counteract hypoglycaemia or predicted hypoglycaemia.

24. The integrated system of any one of claim 22 or 23, wherein:
(i) the system is a subcutaneous system wherein the glucose sensors are subcutaneous and the compound and insulin are administered sub-cutaneously; or
(ii) the system is an intraperitoneal system wherein the glucose sensors are intraperitoneal and the compound and insulin are administered intraperitoneally; or
(iii) the system is a combined subcutaneous/intraperitoneal system, wherein the glucose sensors are sub-cutaneous, the compound is administered sub-cutaneously, and insulin is administered intraperitoneally, wherein optionally compound is administered either intraperitoneally in conjunction with the insulin administration or sub-cutaneously at the glucose sensor site.

25. The integrated system of any one of claims 22 to 24, wherein the compound delivery means is configured to administer compound to a site within 3 cm of the glucose sensor and/or the insulin administration site.

26. A sensor system for determining the level of glucose in the blood of a subject, said sensor system comprising:
(i) a glucose sensor configured to determine the blood glucose level of glucose of the subject and to provide sensor data associated with the blood glucose level;
(ii) a delivery means configured to administer a compound with glucagon activity to said subject;
(iii) a control system configured to receive the sensor data from the glucose sensor; wherein:
(a) the delivery means comprises a slow-release reservoir of the compound with glucagon activity, which is configured to administer the compound to a site in the vicinity of the glucose sensor, thereby to improve blood flow to the vicinity of the glucose sensor; or
(b) the delivery means is controllable to administer the compound with glucagon activity to said subject, and the control system is configured to control the delivery means to administer the compound to a site in the vicinity of the glucose sensor in temporal coordination with operation of the glucose sensor to measure the blood glucose level, thereby to improve blood flow to the vicinity of the glucose sensor.

27. An insulin delivery system for the administration of insulin to a subject, said delivery system comprising:
(i) a compound delivery means configured to administer a compound with glucagon activity to said subject;
(ii) an insulin delivery device configured to administer insulin to said subject; and
(iii) a control system configured to determine a dose of insulin to administer to the subject and to control the insulin delivery device to administer said dose, wherein:
(a) the compound delivery means comprises a slow-release reservoir of the compound with glucagon activity, which is configured to administer the compound to a site in the vicinity of the insulin administration site; or
(b) the compound delivery means is controllable to administer the compound with glucagon activity to said subject, and the control system is configured to control the compound delivery means to administer the compound to a site in the vicinity of the insulin administration site in temporal coordination with the administration of the insulin.
